(19) United States
(12) Patent Application Publication
(10) Pub. No.: US 2015/0025110 A1
(43) Pub. Date: Jan. 22, 2015

(54) 1-CYCLOALKYL- OR 1-HETEROCYCLYL-HYDROXYIMINO-3-PHENYL-PROPANES

(71) Applicant: Hoffmann-La Roche Inc., Nutley, NJ (US)

(72) Inventors: Henrietta Dehmlow, Loerrach (DE); Rainer E. Martin, Basel (CH); Patrizio Mattei, Riehen (CH); Ulrike Obst Sander, Reinach BL (CH); Hans Richter, Grenzach-Wyhlen (DE)

(21) Appl. No.: 14/510,834

(22) Filed: Oct. 9, 2014

Related U.S. Application Data
(63) Continuation of application No. 13/658,878, filed on Oct. 24, 2012, now abandoned.

(30) Foreign Application Priority Data
Oct. 26, 2011 (EP) 11186682.8

(51) Int. Cl. C07D 401/10; C07C 317/32; C07D 335/02; C07C 251/42; C07D 211/76; C07D 309/04

(52) U.S. Cl. CPC C07D 401/10; C07D 211/76; C07D 309/04; C07D 335/02; C07C 251/42; C07C 317/32
USPC 514/316; 546/216; 514/327; 546/188; 546/221; 549/426; 514/459; 549/13; 514/432; 549/28; 564/300; 514/640

(57) ABSTRACT

This invention relates to novel 1-cycloalkyl- or 1-heterocyclyl-hydroxyimino-3-phenyl-propanes of the formula

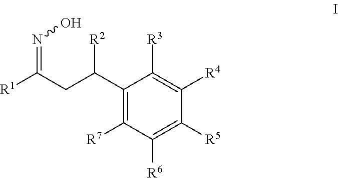

wherein R¹ to R⁷ are as defined in the description and in the claims, as well as pharmaceutically acceptable salts thereof. These compounds are GPBAR1 agonists and may therefore be useful as medicaments for the treatment of diseases such as type II diabetes.

### PRIORITY TO RELATED APPLICATION

This application claims the benefit of European Patent Application No. 11186682.8, filed Oct. 26, 2011, which is hereby incorporated by reference in its entirety.

### FIELD OF THE INVENTION

The present invention relates to novel 1-cycloalkyl- or 1-heterocyclyl-hydroxyimino-3-phenyl-propanes having pharmaceutical activity, their manufacture, pharmaceutical compositions containing them and their potential use as medicaments.

In particular, the present invention relates to compounds of the formula

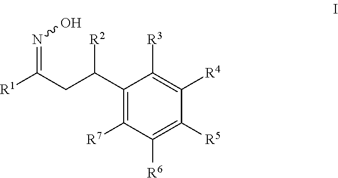

wherein R¹ to R⁷ are as described below, or to pharmaceutically acceptable salts thereof.

The compounds are modulators or ligands of the GPBAR1 receptor. More particularly, the compounds are potent GPBAR1 agonists and may be useful for the treatment and prevention of metabolic and inflammatory diseases, in particular type II diabetes.

### BACKGROUND OF THE INVENTION

Diabetes mellitus is an ever-increasing threat to human health. For example, in the United States current estimates maintain that about 16 million people suffer from diabetes mellitus. Type H diabetes also known as non-insulin-dependent diabetes mellitus accounts for approximately 90-95% of diabetes cases, killing about 193,000 U.S. residents each year. Type II diabetes is the seventh leading cause of all deaths. In Western societies, type II diabetes currently affects 6% of the adult population with world-wide frequency expected to grow by 6% per annum. Although there are certain inheritable traits that may predispose particular individuals to developing type II diabetes, the driving force behind the current increase in incidence of the disease is the increased sedentary life-style, diet, and obesity now prevalent in developed countries. About 80% of diabetics with type II diabetes are significantly overweight. Also, an increasing number of young people are developing the disease. Type II diabetes is now internationally recognized as one of the major threats to human health in the 21st century.

Type II diabetes manifests as inability to adequately regulate blood-glucose levels and may be characterized by a defect in insulin secretion or by insulin resistance. Namely, those who suffer from Type II diabetes have too little insulin or cannot use insulin effectively. Insulin resistance refers to the inability of the body tissues to respond properly to endogenous insulin. Insulin resistance develops because of multiple factors, including genetics, obesity, increasing age, and having high blood sugar over long periods of time. Type II diabetes, sometimes called mature on set, can develop at any age, but most commonly becomes apparent during adulthood. However, the incidence of type II diabetes in children is rising. In diabetics glucose levels build up in the blood and urine causing excessive urination, thirst, hunger, and problems with fat and protein metabolism. If left untreated, diabetes mellitus may cause life-threatening complications, including blindness, kidney failure, and heart disease.

Type II diabetes is currently treated at several levels. A first level of therapy is through diet and/or exercise, either alone or in combination with therapeutic agents. Such agents may include insulin or pharmaceuticals that lower blood glucose levels. About 49% of individuals with Type II diabetes require oral medications, about 40% require insulin injections or a combination of insulin injections and oral medications, and 10% use diet and exercise alone.

Current therapies include: insulin secretagogues, such as sulfonylureas, which increase insulin production from pancreatic β-cells; glucose-lowering effectors, such as metformin which reduce glucose production from the liver; activators of the peroxisome proliferator-activated receptor γ (PPARγ), such as the thiazolidinediones, which enhances insulin action; and α-glucosidase inhibitors which interfere with gut glucose production. There are, however, deficiencies associated with currently available treatments. For example sulfonylureas and insulin injections can be associated with hypoglycemic episodes and weight gain. Furthermore, patients often lose responsiveness to sulfonylureas over time. Metformin and α-glucosidase inhibitors often lead to gastrointestinal problems and PPARγ agonists tend to cause increased weight gain and edema.

Bile acids (BA) are amphipathic molecules which are synthesized in the liver from cholesterol and stored in the gall bladder until secretion to the duodenum and intestine to play an important role in the solubilization and absorption of dietary fat and lipid-soluble vitamins. Approx. 99% of BA are absorbed again by passive diffusion and active transport in the terminal ileum and transported back to the liver via the portal vein (enterohepatic circulation). In the liver, BA decrease their own biosynthesis from cholesterol through the activation of the farnesoid X receptor alpha (FXRα) and small heterodimer partner (SHP), leading to the transcriptional repression of cholesterol 7α-hydroxylase, the rate-limiting step of BA biosynthesis from cholesterol.

GPBAR1, in the literature termed TGR5, M-BAR or BG37 as well, was recently identified as a G-protein coupled receptor (GPCR) responsive to BA (Kawamata et al., J. Biol. Chem. 2003, 278, 9435-9440; Maruyama et al., Biochem. Biophys. Res. Commun. 2002, 298, 714-719). GPBAR1 is a G(alpha)s-coupled GPCR and stimulation by ligand binding causes activation of adenylyl cyclase which leads to the elevation of intracellular cAMP and subsequent activation of downstream signalling pathways. The human receptor shares 86, 90, 82, and 83% amino acid identity to bovine, rabbit, rat, and mouse receptor, respectively. GPBAR1 is abundantly expressed in the intestinal tract, monocytes and macrophages, lung, spleen, placenta (Kawamata et al., J. Biol. Chem. 2003, 278, 9435-9440). BA induced receptor internalization, intracellular cAMP production and activation of extracellular signal-regulated kinase in GPBAR1-expressing HEK293 and CHO cells.

GPBAR1 was found to be abundantly expressed in monocytesmacrophages from humans and rabbits (Kawamata et al., J. Biol. Chem. 2003, 278, 9435-9440), and BA treatment suppressed LPS-induced cytokine production in rabbit alveolar macrophages and human THP-1 cells expressing GPBAR1. These data suggest that bile acids can suppress the macrophage function via activation of GPBAR1. In the liver functional GPBAR1 was found in the plasma membranes of Kupffer cells, mediating inhibition of LPS-induced cytokine expression (Keitel, Biochem. Biophys. Res. Commun. 2008, 372, 78-84), and of sinusoidal endothelial cells, where bile salts led to an increase in intracellular cAMP and to the activation and enhanced expression of the endothelial nitric oxide (NO) synthase (Keitel, Hepatology 2007, 45, 695-704). Furthermore, GPBAR1 has been detected in cholangiocytes of rat liver (Keitel, Biochem. Biophys. Res. Commun. 2008, 372, 78-84). Hydrophobic bile acids, such as taurolithocholic acid, increase cAMP in cholangiocytes suggesting that GPBAR1 may modulate ductal secretion and bile flow. Indeed, GPBAR1 staining colocalized with the cyclic adenosine monophosphate regulated chloride channel cystic fibrosis transmembrane conductance regulator (CFTR) and the apical sodium-dependent bile salt uptake transporter (ASBT). A functional coupling of GPBAR1 to chloride secretion and bile flow has been shown using GPBAR1 agonists (Keitel et al., Hepatology 2009 50, 861-870; Pellicciari et al., J Med Chem 2009, 52(24), 7958-7961). In summary, GPBAR1 agonists may trigger a protective as well as medicative mechanism in cholestatic livers.

GPBAR1 is expressed in intestinal enteroendocrine cell lines from human (NCI-H716) and murine (STC-1, GLUTag) origin (Maruyama et al., Biochem. Biophys. Res. Commun. 2002, 298, 714-719). Stimulation of GPBAR1 by BA stimulated cAMP production in NCI-H716 cells. Intracellular increases in cAMP suggested that BA may induce the secretion of glucagon-like peptide-1 (GLP-1). Indeed, activation of GPBAR1 by BA promoted GLP-1 secretion in STC-1 cells (Katsuma et al., Biochem. Biophys. Res. Commun. 2005, 329, 386-390). Receptor-specificity has been demonstrated by RNA interference experiments which revealed that reduced expression of GPBAR1 resulted in diminished secretion of GLP-1. There is compelling evidence that GPBAR1-mediated GLP-1 and PYY release from intestinal L-cells extends to in vivo. In the isolated vascularly perfused rat colon, BAs have been shown to trigger GLP-1 secretion (Plaisancie et al., J. Endocrin. 1995, 145, 521-526). Using a combination of pharmacological and genetic gain- and loss-of-function studies in vivo, GPBAR1 signalling was shown to induce GLP-1 release, leading to improved liver and pancreatic function and enhanced glucose tolerance in obese mice (Thomas et al., Cell Metabolism, 2009, 10, 167-177). In humans, intracolonic administration of deoxycholate showed marked increases in plasma levels of GLP-1 and the co-secreted PYY (Adrian et al., Gut 1993, 34, 1219-1224).

GLP-1 is a peptide secreted from enteroendocrine L cells has been shown to stimulate insulin release in glucose dependent manner in humans (Kreymann et al., Lancet 1987, 2, 1300-1304) and studies in experimental animals demonstrated that this incretin hormone is necessary for normal glucose homeostasis. In addition, GLP-1 can exert several beneficial effects in diabetes and obesity, including 1) increased glucose disposal, 2) suppression in glucose production, 3) reduced gastric emptying, 4) reduction in food intake and 5) weight loss. More recently, much research has been focused on the use of GLP-1 in the treatment of conditions and disorders such as diabetes mellitus, stress, obesity, appetite control and satiety, Alzheimer disease, inflammation, and diseases of the central nervous system. (see, for example, Bojanowska et al., Med. Sci. Monit. 2005, 8, RA271-8; Perry et al., Current Alzheimer Res. 2005, 3, 377-385; and Meier et al., Diabetes Metab. Res. Rev. 2005, 2, 91-117). However, the use of a peptide in clinical treatment is limited due to difficult administration, and in vivo stability. Therefore, a small molecule that either mimics the effects of GLP-1 directly, or increases GLP-1 secretion, may be useful in treatment of the variety of conditions or disorders described above, namely diabetes mellitus.

PYY is co-secreted with GLP-1 from intestinal L-cells following a meal. A dipeptidyl peptidase-IV (DPP4) cleavage product of PYY is PYY[3-36] (Eberlein et al. Peptides 1989, 10, 797-803) (Grandt et al. Regul Pept 1994, 51, 151-159). This fragment constitutes approximately 40% of total PYY-like immunoreactivity in human and canine intestinal extracts and about 36% of total plasma PYY immunoreactivity in a fasting state to slightly over 50% following a meal. PYY[3-36] is reportedly a selective ligand at the Y2 and Y5 receptors. Peripheral administration of PYY reportedly reduces gastric acid secretion, gastric motility, exocrine pancreatic secretion (Yoshinaga et al. Am J Physiol 1992, 263, G695-701), gallbladder contraction and intestinal motility (Savage et al. Gut 1987, 28, 166-170). It has been demonstrated that intra-arcuate (IC) or intra-peritoneal (IP) injection of PYY3-36 reduced feeding in rats and, as a chronic treatment, reduced body weight gain. Intra-venous (IV) infusion (0.8 μmol/kg/min) for 90 min of PYY3-36 reduced food intake in obese and normal human subjects 33% over 24 hours. These finding suggest that the PYY system may be a therapeutic target for the treatment of obesity (Bloom et. al. Nature 2002, 418, 650-654).

Furthermore, activation of GPBAR1 might be beneficial for the treatment of obesity and metabolic syndrome. Mice fed a high fat diet (HFD) containing 0.5% cholic acid gained less weight than control mice on HFD alone independent of food intake (Watanabe et al., Nature 2006, 439, 484-489). These effects were independent of FXR-alpha, and are likely to results from the binding of BA to GPBAR1. The proposed GPBAR1-mediated mechanism is leading to the subsequent induction of the cAMP-dependent thyroid hormone activating enzyme type 2 (D2) which converts the inactive T3 into the active T4, resulting in the stimulation of the thyroid hormone receptor and promoting energy expenditure. Mice lacking the D2 gene were resistant to cholic acid-induced weight loss. In both rodents and humans, the most thermogenically important tissues (the brown adipose and skeletal muscle) are specifically targeted by this mechanism because they co-express D2 and GPBAR1. The BA-GPBAR1-cAMP-D2 signalling pathway is therefore a crucial mechanism for fine-tuning energy homeostasis that can be targeted to improve metabolic control.

It is therefore an object of the present invention to provide selective, directly acting GPBAR1 agonists. Such agonists are useful as therapeutically active substances, particularly in the treatment and/or prevention of diseases which are associated with the activation of GPBAR1.

The novel compounds of the present invention exceed the compounds known in the art, inasmuch as they are small molecules and they bind to and selectively activate GPBAR1 very efficiently. They are expected to have an enhanced therapeutic potential compared to the compounds already known in the art and can be used for the treatment of diabetes, obesity, metabolic syndrome, hypercholesterolemia, dyslipidemia and a wide range of acute and chronic inflammatory diseases.

### SUMMARY OF THE INVENTION

The present invention relates to 1-cycloalkyl- or 1-heterocyclyl-hydroxyimino-3-phenyl-propanes of formula I,

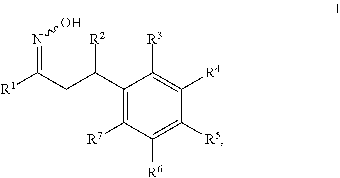

wherein
R¹ is C₄₋₇-cycloalkyl, said cycloalkyl being unsubstituted or substituted by one, two or three groups independently selected from the group consisting of C₁₋₇-alkyl, hydroxy, oxo, dioxo and C₁₋₇-alkylcarbonyl; or
heterocyclyl, said heterocyclyl having 4 to 7 ring atoms, comprising one, two or three heteroatoms selected from N, O and S and being unsubstituted or substituted by one, two or three groups independently selected from the group consisting of C₁₋₇-alkyl, hydroxy, oxo, dioxo, and C₁₋₇-alkylcarbonyl;
R² is selected from the group consisting of C₁₋₇-alkyl,
C₃₋₇-cycloalkyl, C₂₋₇-alkenyl, halogen-C₁₋₇-alkyl,
unsubstituted phenyl or phenyl substituted by one, two or three groups independently selected from the group consisting of C₁₋₇-alkyl, halogen, halogen-C₁₋₇-alkyl, halogen-C₁₋₇-alkoxy and C₁₋₇-alkylsulfonyl, and
heteroaryl, said heteroaryl being unsubstituted or substituted by C₁₋₇-alkyl or oxo, R³ and R⁷ are independently from each other selected from the group consisting of hydrogen, halogen and C₁₋₇-alkyl; and
R⁴, R⁵ and R⁶ are independently selected from the group consisting of hydrogen,
halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkyl sulfonyl, C₁₋₇-alkylsulfonyloxy,
C₁₋₇-alkylsulfonylamino, C₃₋₇-cycloalkylsulfonylamino,
aminosulfonyl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₇-alkyl-amino
hydroxy-C₁₋₇-alkyl-C₁₋₇-alkyl-amino, an amino acid attached through the amino group of the amino acid,
C₃₋₇-cycloalkyl-amino, wherein C₃₋₇-cycloalkyl is unsubstituted or substituted by hydroxy, hydroxy-C₁₋₇-alkyl or carboxyl,
carboxyl-C₁₋₇-alkyl-aminocarbonyl, carboxyl-C₁₋₇-alkyl-(C₁₋₇-alkyl)-aminocarbonyl,
C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl,
hydroxy-C₁₋₇-alkyl-C₁₋₇-alkyl-aminocarbonyl, halogen-hydroxy-C₁₋₇-alkyl-aminocarbonyl,
C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl-C₁₋₇-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₃₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl,
heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahydropyranyl, carboxyl, carboxyl-C₁₋₇-alkyl, C₁₋₇-alkoxy-C₁₋₇-alkyl or C₁₋₇-alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl,
C₁₋₇-alkoxycarbonyl-C₁₋₇-alkyl-carbonylamino-C₁₋₇-alkylsulfonyl, phenyl-C₁₋₇-alkyl-aminocarbonyl, tetrazolyl-aminocarbonyl, tetrazolyl-C₁₋₇-alkyl-aminocarbonyl and carboxyl-C₁₋₇-alkyl-aminocarbonyl;
or pharmaceutically acceptable salts thereof.

The invention is also concerned with processes for the manufacture of compounds of formula I.

The invention also relates to pharmaceutical compositions comprising a compound of formula I as described above and a pharmaceutically acceptable carrier and/or adjuvant.

A further aspect of the invention is the use of compounds of formula I as therapeutic active substances for the treatment of diseases which are associated with the modulation of GPBAR1 activity. The invention thus relates to a method for the treatment of a disease associated with the modulation of GPBAR1 activity such as for example diabetes, particularly type II diabetes or gestational diabetes.

### DETAILED DESCRIPTION OF THE INVENTION

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention.

The nomenclature used in this application is based on IUPAC systematic nomenclature, unless indicated otherwise.

The term “compound(s) of this invention” and “compound(s) of the present invention” refers to compounds of formula I and stereoisomers, solvates or salts thereof (e.g., pharmaceutically acceptable salts).

The term “substituent” denotes an atom or a group of atoms replacing a hydrogen atom on the parent molecule.

The term “halogen” refers to fluoro, chloro, bromo and iodo, with fluoro, chloro and bromo being of particular interest. More particularly, halogen refers to fluoro and chloro.

The term “alkyl”, alone or in combination with other groups, refers to a branched or straight-chain monovalent saturated aliphatic hydrocarbon radical of one to twenty carbon atoms, particularly one to sixteen carbon atoms, more particularly one to ten carbon atoms. The term “C₁₋₁₀-alkyl” refers to a branched or straight-chain monovalent saturated aliphatic hydrocarbon radical of one to ten carbon atoms, such as e.g., methyl, ethyl, n-propyl, isopropyl, n-butyl, sec-butyl, tert-butyl, pentyl, 1,1,3,3-tetramethyl-butyl and the like. More particularly, the term “alkyl” also embraces lower alkyl groups as described below.

The term “lower alkyl” or “C₁₋₇-alkyl”, alone or in combination, signifies a straight-chain or branched-chain alkyl group with 1 to 7 carbon atoms, in particular a straight or branched-chain alkyl group with 1 to 6 carbon atoms and more particularly a straight or branched-chain alkyl group with 1 to 4 carbon atoms. Examples of straight-chain and branched C₁₋₇ alkyl groups are methyl, ethyl, propyl, isopropyl, butyl, isobutyl, tert-butyl, the isomeric pentyls, the isomeric hexyls and the isomeric heptyls, in particular methyl and ethyl.

The term “lower alkenyl” or “C₂₋₄-alkenyl” signifies a straight-chain or branched chain hydrocarbon residue comprising an olefinic bond and 2 to 7, preferably 3 to 6, particularly preferred 3 to 4 carbon atoms. Examples of alkenyl groups are ethenyl, 1-propenyl, 2-propenyl, isopropenyl, 1-butenyl, 2-butenyl, 3-butenyl and isobutenyl, in particular 2-propenyl (allyl).

The term “lower alkynyl” or “C₂₋₇-alkynyl” signifies a straight-chain or branched chain hydrocarbon residue comprising a triple bond and 2 to 7 carbon atoms. Examples of lower alkynyl groups are ethynyl and 1-propynyl (—C≡C—CH₂).

The term “cycloalkyl” or “C₃₋₇-cycloalkyl” denotes a saturated monocyclic hydrocarbon group containing from 3 to 7 carbon atoms, such as cyclopropyl, cyclobutyl, cyclopentyl, cyclohexyl or cycloheptyl, more particularly cyclopropyl. In addition, the term “cycloalkyl” also embraces bicyclic hydrocarbon groups containing from 3 to 10 carbon atoms. Bicyclic means consisting of two saturated carbocycles having one or more carbon atoms in common. Examples for bicyclic cycloalkyl are bicyclo[2.2.1]heptanyl or bicyclo[2.2.2]octanyl.

The term “lower cycloalkylalkyl” or “C₃₋₇-cycloalkyl-C₁₋₇-alkyl” refers to lower alkyl groups as defined above wherein at least one of the hydrogen atoms of the lower alkyl group is replaced by a cycloalkyl group. Among the lower cycloalkylalkyl groups of particular interest resides cyclopropylmethyl.

The term “lower alkoxy” or “C₁₋₇-alkoxy” refers to the group R′—O—, wherein R′ is lower alkyl and the term “lower alkyl” has the previously given significance. Examples of lower alkoxy groups are methoxy, ethoxy, n-propoxy, isopropoxy, n-butoxy, isobutoxy, sec.-butoxy and tert-butoxy, in particular methoxy.

The term “lower alkoxyalkyl” or “C₁₋₇-alkoxy-C₁₋₇-alkyl” refers to lower alkyl groups as defined above wherein at least one of the hydrogen atoms of the lower alkyl group is replaced by a lower alkoxy group. Among the lower alkoxyalkyl groups of particular interest are methoxymethyl and 2-methoxyethyl.

The term “lower alkoxyalkoxyalkyl” or “C₁₋₇-alkoxy-C₁₋₇-alkoxy-C₁₋₇-alkyl” refers to lower alkyl groups as defined above wherein at least one of the hydrogen atoms of the lower alkyl group is replaced by a lower alkoxy group which itself is also substituted by a further lower alkoxy group. Among the lower alkoxyalkoxyalkyl groups of particular interest is —(CH₂)₂—O—(CH₂)₂—O—CH₃.

The term hydroxy means the group —OH.

The term “lower hydroxyalkyl” or “hydroxy-C₁₋₇-alkyl” refers to lower alkyl groups as defined above wherein at least one of the hydrogen atoms of the lower alkyl group is replaced by a hydroxy group. Among the particular interesting lower hydroxyalkyl groups are hydroxymethyl or hydroxyethyl.

The term “lower hydroxyalkenyl” or “hydroxy-C₁₋₇-alkenyl” refers to lower alkenyl groups as defined above wherein at least one of the hydrogen atoms of the lower alkenyl group is replaced by a hydroxy group. Among the particular interesting lower hydroxyalkenyl groups is 3-hydroxy-propenyl.

The term “lower hydroxyalkynyl” or “hydroxy-C₁₋₇-alkynyl” refers to lower alkynyl groups as defined above wherein at least one of the hydrogen atoms of the lower alkynyl group is replaced by a hydroxy group. Among the particular interesting lower hydroxyalkynyl groups is 3-hydroxy-propinyl.

The term “lower halogenalkyl” or “halogen-C₁₋₇-alkyl” refers to lower alkyl groups as defined above wherein at least one of the hydrogen atoms of the lower alkyl group is replaced by a halogen atom, particularly fluoro or chloro, most particularly fluoro. Among the lower halogenalkyl groups of particular interest are trifluoromethyl, difluoromethyl, trifluoroethyl, 2,2-difluoroethyl, fluoromethyl and chloromethyl, with trifluoromethyl or difluoromethyl being especially interesting.

The term “lower halogenalkoxy” or “halogen-C₁₋₇-alkoxy” refers to lower alkoxy groups as defined above wherein at least one of the hydrogen atoms of the lower alkoxy group is replaced by a halogen atom, particularly fluoro or chloro, most particularly fluoro. Among the lower halogenalkoxy groups of particular interest are trifluoromethoxy, difluoromethoxy, fluoromethoxy and chloromethoxy, more particularly trifluoromethoxy.

The term “carboxyl” means the group —COOH.

The term “lower carboxylalkyl” or “carboxyl-C₁₋₇-alkyl” refers to lower alkyl groups as defined above wherein at least one of the hydrogen atoms of the lower alkyl group is replaced by a carboxyl group. Among the lower carboxylalkyl groups or particular interest are carboxylmethyl (—CH₂—COOH) and carboxylethyl (—CH₂—CH₂—COOH).

The term “lower carboxylalkenyl” or “carboxyl-C₁₋₇-alkenyl” refers to lower alkenyl groups as defined above wherein at least one of the hydrogen atoms of the lower alkenyl group is replaced by a carboxyl group. Among the particular interesting lowercarboxylalkenyl groups is 3-carboxyl-propenyl (—CH═CH—CH₂—COOH).

The term “lower carboxylalkynyl” or “carboxyl-C₁₋₇-alkynyl” refers to lower alkynyl groups as defined above wherein at least one of the hydrogen atoms of the lower alkynyl group is replaced by a carboxyl group. Among the particular interesting lower carboxylalkynyl groups is 3-carboxyl-propinyl.

The term “lower carboxylalkoxy” or “carboxyl-C₁₋₇-alkoxy” refers to lower alkoxy groups as defined above wherein at least one of the hydrogen atoms of the lower alkoxy group is replaced by a carboxyl group. A lower carboxylalkoxy group of particular interest is carboxylmethoxy (—O—CH₂—COOH).

The term “lower carboxylalkylaminocarbonyl” or “carboxyl-C₁₋₇-alkylaminocarbonyl” refers to aminocarbonyl as defined above wherein one of the hydrogen atoms of the amino group is replaced by carboxyl-C₁₋₇-alkyl. Preferred lower carboxylalkylaminocarbonyl group is —CO—NH—CH₂—COOH.

The term “lower alkoxycarbonyl” or “C₁₋₇-alkoxycarbonyl” refers to the group —COOR, wherein R is lower alkyl and the term “lower alkyl” has the previously given significance. Lower alkoxycarbonyl groups of particular interest are methoxycarbonyl or ethoxycarbonyl.

The term “lower alkoxycarbonylalkyl” or “C₁₋₇-alkoxycarbonyl-C₁₋₇-alkyl” means lower alkyl groups as defined above wherein one of the hydrogen atoms of the lower alkyl group is replaced by C₁₋₇-alkoxycarbonyl. A particular lower alkoxycarbonylalkyl group is —CH₂—COOCH₃.

The term “di-(lower alkoxycarbonyl)-alkyl” or “di-(C₁₋₇-alkoxycarbonyl)-C₁₋₇-alkyl” means lower alkyl groups as defined above wherein two of the hydrogen atoms of the lower alkyl group are replaced by C₁₋₇-alkoxycarbonyl. A particular di-(lower alkoxycarbonyl)-alkyl group is —CH—(COOCH₃)₂.

The term “lower alkoxycarbonylalkoxy” or “C₁₋₇-alkoxycarbonyl-C₁₋₇-alkoxy” means a lower alkoxy group as defined above wherein one of the hydrogen atoms of the lower alkoxy group is replaced by C₁₋₇-alkoxycarbonyl. An example for a lower alkoxycarbonylalkoxy group is O—CH₂—COOCH₃.

The term “lower alkoxycarbonylalkylaminocarbonyl” or “C₁₋₇-alkoxycarbonyl-C₁₋₇-alkylaminocarbonyl” refers to aminocarbonyl as defined above wherein one of the hydrogen atoms of the amino group is replaced by C₁₋₇-alkoxycarbonyl-C₁₋₇-alkyl. Preferred lower alkoxycarbonylalkylaminocarbonyl group is CO—NH—CH₂—COOCH₃.

The term “lower alkylsulfonyl” or “C₁₋₇-alkylsulfonyl” means the group —S(O)₂—R, wherein R is a lower alkyl group as defined above. A lower alkylsulfonyl group of particular interest is methylsulfonyl.

The term “lower alkylcarbonyl” or “C₁₋₇-alkylcarbonyl” means the group —C(O)—R, wherein R is a lower alkyl group as defined above. A lower alkylcarbonyl group of particular interest is methylcarbonyl or acetyl.

The term “C₁₋₇-alkylsulfonyloxy” means the group —O—S(O)₂—R, wherein R is a lower alkyl group as defined above.

The term “aminosulfonyl” means the group —S(O)₂—NH₂.

The term “lower alkylaminosulfonyl” or “C₁₋₇-alkyl-aminosulfonyl” defines the group —S(O)₂—NH—R, wherein R is lower alkyl and the term “lower alkyl” has the previously given meaning. An example of a lower alkylaminosulfonyl group is methylaminosulfonyl.

The term “di-lower alkylaminosulfonyl” or “di-(C₁₋₇-alkyl)-aminosulfonyl” defines the group —S(O)₂—NRR′, wherein R and R′ are lower alkyl groups as defined above. An example of a di-lower alkylaminosulfonyl group is dimethylaminosulfonyl.

The term “heterocyclylsulfonyl” defines a group —S(O)₂-Het, wherein Het is a heterocyclyl group as defined herein below.

“Amino” refers to the group —NH₂. The term “C₁₋₇-alkylamino” means a group —NHR, wherein R is lower alkyl and the term “lower alkyl” has the previously given significance. The term “di-(C₁₋₇-alkyl)-amino” means a group —NRR′, wherein R and R′ are lower alkyl groups as defined above.

The term “C₁₋₇-alkoxy-C₁₋₇-alkyl-C₁₋₇-alkylamino” refers to a group —NRR″, wherein R is a lower alkyl group as defined above and R″ is a lower alkoxyalkyl group as defined herein.

The term “C₁₋₇-hydroxyalkyl-C₁₋₇-alkylamino” refers to a group —NRR′″, wherein R is a lower alkyl group as defined above and R′″ is a lower hydroxyalkyl group as defined herein.

The term “C₁₋₇-alkoxy-halogen-C₁₋₇-alkyl-amino” refers to a group —NR^{x}R^{y}, wherein R^{x} is a lower alkyl group as defined above and R^{y} is a lower halogenalkyl group as defined herein.

The term “cycloalkyl-amino” or “C₃₋₇-cycloalkyl-amino” means a group —NH—R^{C}, wherein R^{C} is a cycloalkyl group as defined above.

The term “carboxylalkyl-alkylamino” or “carboxyl-C₁₋₇-alkyl-C₁₋₇-alkyl-amino” defines the group —NR—R^{B}, wherein R is lower alkyl as defined above and R^{B} is lower carboxylalkyl and has the previously given meaning.

The term “lower alkylsulfonylamino” or “C₁₋₇-alkylsulfonylamino” defines the group —NH—S(O)₂—R, wherein R is lower alkyl and the term “lower alkyl” has the previously given meaning.

The term “cycloalkylsulfonylamino” or “C₃₋₄-cycloalkylsulfonylamino” defines the group

—NH—S(O)₂—R^{C}, wherein R^{C} is cycloalkyl and has the previously given meaning. An example is clyclopropylsulfonylamino.

The term “lower alkylcarbonylamino” or “C₁₋₇-alkylcarbonylamino” defines the group —NH—CO—R, wherein R is lower alkyl and the term “lower alkyl” has the previously given meaning.

The term “lower carboxylalkylcarbonylamino” or “carboxyl-C₁₋₇-alkylcarbonylamino” defines the group —NH—CO—R^{B}, wherein R^{B} is lower carboxylalkyl and has the previously given meaning.

The term “lower alkoxycarbonyl-carbonylamino” or “C₁₋₇-alkoxycarbonyl-carbonylamino” defines the group —NH—CO—R^{E}, wherein R^{E} is lower alkoxycarbonyl and has the previously given meaning.

The term “lower alkoxycarbonyl-alkylcarbonylamino” or “C₁₋₇-alkoxycarbonyl-C₁₋₇-alkylcarbonylamino” defines the group —NH—CO—R—R^{E}, wherein R is a lower alkyl group as defined above and at least one of the hydrogen atoms of the lower alkyl group is replaced by a lower alkoxycarbonyl group R^{E} as defined above.

The term “lower alkoxycarbonyl-alkylcarbonylamino-alkylsulfonyl” or “C₁₋₇-alkoxycarbortyl-C₁₋₇-alkyl-carbonylamino-C₁₋₇-alkylsulfonyl” refers to the group —S(O)₂—R—NH—CO—R′—R^{E}, wherein R and R′ are lower alkyl groups as defined above and at least one of the hydrogen atoms of the lower alkyl group R′ is replaced by a lower alkoxycarbonyl group R^{E} as defined above.

The term “an amino acid attached through the amino group of the amino acid” means the substituent —NR—CHR^{A}—COOH, wherein R is hydrogen or lower alkyl as defined above and R^{A} is the side chain of an amino acid, in particular the side chain of a natural amino acid, but R^{A} denotes also other organic substituents such as chloromethyl.

The term “aminocarbonyl” refers to the group —CO—NH₂.

The term “lower alkylaminocarbonyl” or “C₁₋₇-alkyl-aminocarbonyl” refers to a group —CONH—R, wherein R is lower alkyl as defined herein before.

The term “lower dialkylaminocarbonyl” or “di-(C₁₋₇-alkyl)-aminocarbonyl” refers to a group —CONRR′, wherein R and R′ are lower alkyl groups as defined above.

The term “lower alkylsulfonyl-lower alkylaminocarbonyl” or “C₁₋₇-alkylsulfonyl-C₁₋₇-alkyl-aminocarbonyl” refers to a group —CONR—R^{S}, wherein R is lower alkyl as defined herein before and R^{S} is a lower alkylsulfonyl group as defined above.

The term “hydroxysulfonyl” means the group —S(O)₂—OH.

The term “lower hydroxysulfonylalkyl-aminocarbonyl” or “hydroxysulfonyl-C₁₋₇-alkyl-aminocarbonyl” means a group —CONH—R^{W}, wherein R^{W} is a lower alkyl group as defined above and wherein one of the hydrogen atoms of the lower alkyl group is replaced by —S(O)₂—OH. An example is —CONH—CH₂—CH₂—S(O)₂—OH.

The term “lower aminocarbonylalkyl” or “aminocarbonyl-C₁₋₇-alkyl” means lower alkyl groups as defined above wherein one of the hydrogen atoms of the lower alkyl group is replaced by aminocarbonyl. A lower aminocarbonylalkyl group of particular interest is —CH₂—CONH₂.

The term “lower halogenalkyl-aminocarbonyl” or “halogen-C₁₋₇-alkyl-aminocarbonyl” refers to a group —CONH—R^{y}, wherein R^{y} is a lower halogenalkyl group as defined above.

The term “lower hydroxyalkyl-aminocarbonyl” or “hydroxy-C₁₋₇-alkyl-aminocarbonyl” means a group —CONH—R′″, wherein R′″ is a lower hydroxyalkyl group as defined above.

The term “lower hydroxyalkyl-aminocarbonylalkyl” or “hydroxy-C₁₋₇-alkyl-aminocarbonyl-C₁₋₇-alkyl” denotes a lower alkyl group as defined above wherein one of the hydrogen atoms of the lower alkyl group is replaced by a group —CONH—R′″, wherein R′″ is a lower hydroxyalkyl group as defined above.

The term “lower halogenhydroxyalkyl-aminocarbonyl” or “halogen-hydroxy-C₁₋₇-alkyl-aminocarbonyl” means a group —CONH—R^{N}, wherein R^{N} is a lower hydroxyalkyl group as defined above and wherein at least one of the hydrogen atoms of the lower hydroxyalkyl group is replaced by a halogen atom, particularly fluoro or chloro.

The term “(lower hydroxyalkyl)-lower alkylaminocarbonyl” or “hydroxy-C₁₋₇-alkyl-C₁₋₇-alkylaminocarbonyl” means a group —CONR—R′″, wherein R is a lower alkyl group as defined herein before, in particular methyl, and R′″ is a lower hydroxyalkyl group as defined above.

The term “lower alkoxyalkyl-aminocarbonyl” or “(C₁₋₇-alkoxy-C₁₋₇-alkyl)-aminocarbonyl” means a group —CONH—R^{Z}, wherein R^{Z} is a lower alkoxyalkyl group as defined above.

The term “cycloalkyl-aminocarbonyl” or “C₃₋₇-cycloalkyl-aminocarbonyl” means a group —CONH—R^{C}, wherein R^{C} is a cycloalkyl group as defined above.

The term “lower carboxylalkyl-aminocarbonyl” or “carboxyl-C₁₋₇-alkyl-aminocarbonyl” means a group —CONH—R^{D}, wherein R^{D} is a lower carboxylalkyl group as defined above, for example —CONH—CH₂—COOH.

The term “lower alkoxycarbonyl-alkyl-aminocarbonyl” or “C₁₋₇-alkoxycarbonyl-C₁₋₇-alkyl-aminocarbonyl” defines the group —CO—NH—R—R^{E}, wherein R is a lower alkyl group as defined above and at least one of the hydrogen atoms of the lower alkyl group is replaced by a lower alkoxycarbonyl group as defined above.

The term “heterocyclyl-aminocarbonyl” means a group —CONH-Het, wherein Het is a heterocyclyl group as defined herein below.

The term “lower heterocyclylalkyl-aminocarbonyl” or “heterocyclyl-C₁₋₇-alkyl-aminocarbonyl” refers to a group —CONH—R^{H}, wherein R^{H} is a lower alkyl group as defined above wherein at least one of the hydrogen atoms of the lower alkyl group is replaced by a heterocyclyl group as defined herein below.

The term “lower alkylcarbonylamino-alkylaminocarbonyl” or “C₁₋₇-alkylcarbonylamino-C₁₋₇-alkylaminocarbonyl” refers to aminocarbonyl as defined above wherein one of the hydrogen atoms of the amino group is replaced by C₁₋₇-alkylcarbonylamino-C₁₋₇-alkyl. An example for a lower alkylcarbonylamino-alkylaminocarbonyl group is —CO—NH—CH₂—CH₂—NH—CO—CH₃.

The term “phenyloxy” refers to the group —O-Ph wherein Ph is phenyl.

The term “lower phenylalkyl” or “phenyl-C₁₋₇-alkyl” means lower alkyl groups as defined above wherein one of the hydrogen atoms of the lower alkyl group is replaced by an optionally substituted phenyl group.

The term “lower phenylalkyl-aminocarbonyl” or “(phenyl-C₁₋₇-alkyl)-aminocarbonyl” means a group —CONH—R^{V}, wherein R^{V} is a lower phenylalkyl group as defined above.

The term “heterocyclyl” refers to a saturated or partly unsaturated monocyclic or bicyclic ring containing from 3 to 10 ring atoms which can comprise one, two or three atoms selected from nitrogen, oxygen and/or sulfur. Bicyclic means consisting of two cycles having two ring atoms in common, i.e. the bridge separating the two rings is either a single bond or a chain of one or two ring atoms. Examples of monocyclic heterocyclyl rings containing in particular from 3 to 7 ring atoms include azirinyl, azetidinyl, oxetanyl, piperidinyl, piperazinyl, azepinyl, diazepanyl, pyrrolidinyl, pyrazolidinyl, imidazolinyl, imidazolidinyl, pyridinyl, pyridazinyl, pyrimidinyl, oxazolidinyl, isoxazolidinyl, morpholinyl, thiazolidinyl, isothiazolidinyl, thiadiazolidinyl, dihydrofuryl, tetrahydrofuryl, tetrahydropyranyl, tetrahydrothiopyranyl, 1,1-dioxo-hexahydro-1,6-thiopyranyl, thiomorpholinyl and 1,1-dioxo-1λ6-thiomorpholinyl. Examples of bicyclic heterocyclyl rings are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl and 3-thia-9-aza-bicyclo[3.3.1]nonyl. Examples for partly unsaturated heterocyclyl are dihydrofuryl, imidazolinyl, dihydro-oxazolyl, tetrahydro-pyridinyl, or dihydropyranyl.

The term “lower heterocyclylalkyl” or “heterocyclyl-C₁₋₇-alkyl” refers to lower alkyl groups as defined above wherein at least one of the hydrogen atoms of the lower alkyl group is replaced by a heterocyclyl group as defined above.

The term “heterocyclylcarbonyl” refers to the group —CO-Het wherein Het is a heterocyclyl group as defined above.

The term “heteroaryl” in general refers to an aromatic 5- or 6-membered ring which comprises one, two, three or four atoms selected from nitrogen, oxygen and/or sulfur, such as pyridyl, pyrazinyl, pyrimidinyl, 2,4-dioxo-1H-pyrimidinyl, pyridazinyl, 2-oxo-1,2-dihydropyridinyl, pyrrolyl, oxazolyl, oxadiazolyl, isoxazolyl, thiadiazolyl, tetrazolyl, pyrazolyl, imidazolyl, furanyl, thiazolyl, isothiazolyl, triazolyl, tetrazolyl, thienyl, azepinyl, diazepinyl. The term “heteroaryl” further refers to bicyclic aromatic groups comprising from 5 to 12 ring atoms, in which one or both rings can contain one, two or three atoms selected from nitrogen, oxygen or sulfur, such as quinolinyl, isoquinolinyl, cinnolinyl, quinazolinyl, pyrazolo[1,5-a]pyridyl, imidazo[1,2-a]pyridyl, quinoxalinyl, benzofuranyl, benzothienyl, benzothiazolyl, benzotriazolyl, indolyl and indazolyl.

The term “lower heteroarylalkyl” or “heteroaryl-C₁₋₇-alkyl” refers to lower alkyl groups as defined above wherein at least one of the hydrogen atoms of the lower alkyl group is replaced by a heteroaryl group as defined above. A specific example of a lower heteroarylalkyl group is tetrazolyl-C₁₋₇-alkyl.

The term “heteroaryl-aminocarbonyl” means a group —CONH—R^{U}, wherein R^{U} is a heteroaryl group as defined above. A specific example of a heteroaryl-aminocarbonyl group is tetrazolylaminocarbonyl.

The term “oxo” means that a C-atom of the heterocyclyl or heteroaryl ring may be substituted by ═O, thus meaning that the heterocyclyl or heteroaryl ring may contain one or more carbonyl (—CO—) groups.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

Compounds of formula I can form pharmaceutically acceptable salts. The term “pharmaceutically acceptable salts” refers to those salts which retain the biological effectiveness and properties of the free bases or free acids, which are not biologically or otherwise undesirable. Pharmaceutically acceptable salts include both acid and base addition salts. The salts are for example acid addition salts of compounds of formula I with physiologically compatible mineral acids, such as hydrochloric acid, hydrobromic acid, nitric acid, carbonic acid, sulfuric acid, sulfurous acid or phosphoric acid; or with organic acids, such as methanesulfonic acid, ethanesulfonic acid, p-toluenesulfonic acid, formic acid, acetic acid, propionic acid, glycolic acid, pyruvic acid, oxylic acid, lactic acid, trifluoroacetic acid, citric acid, fumaric acid, maleic acid, malonic acid, tartaric acid, benzoic acid, cinnamic acid, mandelic acid, embonic acid, succinic acid or salicylic acid. In addition, pharmaceutically acceptable salts may be prepared from addition of an inorganic base or an organic base to the free acid. Salts derived from an inorganic base include, but are not limited to, the sodium, potassium, lithium, ammonium, calcium, magnesium, zinc, copper, manganese and aluminium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, histidine, caffeine, procaine, hydrabamine, choline, betaine, ethylendiamine, glucosamine, methylglucamine, theobromine, piperazine, N-ethylpiperidine, piperidine and polyamine resins. The compound of formula I can also be present in the form of zwitterions. Pharmaceutically acceptable salts of compounds of formula I of particular interest are the sodium salts or salts with tertiary amines.

The compounds of formula I can also be solvated, e.g., hydrated. The solvation can be effected in the course of the manufacturing process or can take place e.g. as a consequence of hygroscopic properties of an initially anhydrous compound of formula I (hydration). The term “pharmaceutically acceptable salts” also includes physiologically acceptable solvates.

“Isomers” are compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”. Diastereomers have two or more chiral centers and are characterized by different physical properties, e.g. melting points, boiling points, spectral properties, and reactivities. Stereoisomers that are non-superimposable mirror images are termed “enantiomers”, or sometimes optical isomers. A carbon atom bonded to four non-identical substituents is termed a “chiral center”.

The term “modulator” denotes a molecule that interacts with a target. The interactions include e.g. agonistic, antagonistic, or inverse agonistic activity.

The term “agonist” denotes a compound that enhances the activity of another compound or receptor site as defined e.g. in Goodman and Gilman's “The Pharmacological Basis of Therapeutics, 7th ed.” in page 35, Macmillan Publ. Company, Canada, 1985. A “full agonist” effects a full response whereas a “partial agonist” effects less than full activation even when occupying the total receptor population. An “inverse agonist” produces an effect opposite to that of an agonist, yet binds to the same receptor binding-site.

The term “half maximal effective concentration” (EC₅₀) denotes the plasma concentration of a particular compound required for obtaining 50% of the maximum of a particular effect in vivo.

The term “therapeutically effective amount” denotes an amount of a compound of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, disease state being treated, the severity or the disease treated, the age and relative health of the subject, the route and form of administration, the judgment of the attending medical or veterinary practitioner, and other factors.

In detail, the present invention relates to compounds of the formula

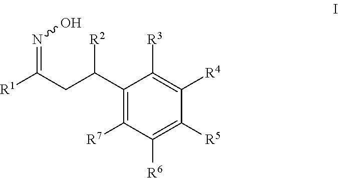

wherein
R¹ is C₄₋₇-cycloalkyl, said cycloalkyl being unsubstituted or substituted by one, two or three groups independently selected from the group consisting of C₁₋₇-alkyl, hydroxy, oxo, dioxo, and C₁₋₇-alkylcarbonyl; or
heterocyclyl, said heterocyclyl having 4 to 7 ring atoms, comprising one, two or three heteroatoms selected from N, O and S and being unsubstituted or substituted by one, two or three groups independently selected from the group consisting of C₁₋₇-alkyl, hydroxy, oxo, dioxo, and C₁₋₇-alkylcarbonyl;
R² is selected from the group consisting of C₁₋₇-alkyl,
C₃₋₇-cycloalkyl, C₂₋₇-alkenyl, halogen-C₁₋₇-alkyl,
unsubstituted phenyl or phenyl substituted by one, two or three groups independently selected from the group consisting of C₁₋₇-alkyl, halogen, halogen-C₁₋₇-alkyl, halogen-C₁₋₇-alkoxy and C₁₋₇-alkylsulfonyl, and
heteroaryl, said heteroaryl being unsubstituted or substituted by C₁₋₇-alkyl or oxo,
R³ and R⁷ are independently from each other selected from the group consisting of hydrogen, halogen and C₁₋₇-alkyl; and
R⁴, R⁵ and R⁶ are independently selected from the group consisting of
hydrogen,
halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkylsulfonyl, C₁₋₇-alkylsulfonyloxy,
C₁₋₇-alkylsulfonylamino, C₃₋₇-cycloalkylsulfonylamino,
aminosulfonyl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₇-alkyl-amino, hydroxy-C₁₋₇-alkyl-C₁₋₇-alkyl-amino, an amino acid attached through the amino group of the amino acid,
C₃₋₇-cycloalkyl-amino, wherein C₃₋₇-cycloalkyl is unsubstituted or substituted by hydroxy,
hydroxy-C₁₋₇-alkyl or carboxyl,
carboxyl-C₁₋₇-alkyl-aminocarbonyl, carboxyl-C₁₋₇-alkyl-(C₁₋₇-alkyl)-aminocarbonyl,
C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl,
hydroxy-C₁₋₇-alkyl-C₁₋₇-alkyl-aminocarbonyl, halogen-hydroxy-C₁₋₇-alkyl-aminocarbonyl,
C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl-C₁₋₄-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₃₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahydropyranyl, carboxyl, carboxyl-C₁₋₇-alkyl, C₁₋₇-alkoxy-C₁₋₇-alkyl or C₁₋₇-alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl, di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl, C₁₋₇-alkoxycarbonyl-C₁₋₇-alkyl-carbonylamino-C₁₋₇-alkylsulfonyl, phenyl-C₁₋₇-alkyl-aminocarbonyl, tetrazolyl-aminocarbonyl, tetrazolyl-C₁₋₇-alkyl-aminocarbonyl and carboxyl-C₁₋₇-alkyl-aminocarbonyl;
or pharmaceutically acceptable salts thereof.

In one aspect, the invention relates to compounds of formula I according to the invention, wherein R¹ is heterocyclyl, said heterocyclyl having 4 to 7 ring atoms and comprising one, two or three heteroatoms selected from N, O and S and being unsubstituted or substituted by one, two or three groups independently selected from the group consisting of C₁₋₇-alkyl, hydroxy, oxo, dioxo and C₁₋₇-alkylcarbonyl. Particularly, R¹ is a heterocyclyl having 6 ring atoms and comprising one heteroatom selected from N, O and S.

In particular, the invention relates to compounds of formula I, wherein R¹ is heterocyclyl, said heterocyclyl being selected from the group consisting of piperidinyl, tetrahydropyranyl and tetrahydrothiopyranyl and being unsubstituted or substituted by one, two or three groups independently selected from the group consisting of C₁₋₇-alkyl, hydroxy, oxo, dioxo, and C₁₋₇-alkylcarbonyl. More particularly, R¹ is selected from 1-methyl-2-oxo-piperidin-3-yl, 1-acetyl-piperidin-4-yl, tetrahydro-2H-pyran-4-yl, tetrahydro-2H-thiopyran-4-yl and 1,1-dioxo-hexahydro-thiopyran-4-yl. Most particularly, R¹ is 1-methyl-2-oxo-piperidin-3-yl.

In another aspect, the invention relates to compounds of formula I according to the invention, wherein R¹ is C₄₋₇-cycloalkyl, said cycloalkyl being unsubstituted or substituted by one, two or three groups independently selected from the group consisting of C₁₋₇-alkyl, hydroxy, oxo, dioxo, and C₁₋₇-alkylcarbonyl. In particular, the invention relates to compounds of formula I, wherein R¹ is C₄₋₇-cycloalkyl, said cycloalkyl being substituted by one, two or three groups independently selected from the group consisting of C₁₋₇-alkyl, hydroxy, oxo, dioxo, and C₁₋₇-alkylcarbonyl. More particularly, R¹ is C₄₋₇-cycloalkyl, said cycloalkyl being substituted by one, two or three groups independently selected from the group consisting of C₁₋₇-alkyl, hydroxyl and oxo. Even more particularly, R¹ is selected from 3-hydroxycyclobutyl, 4-hydroxycyclohexyl, 4-oxocyclohexyl and 4-hydroxy-4-methylcyclohexyl.

Thus, the invention relates in particular to compounds of formula I, wherein R¹ is selected from 1-methyl-2-oxo-piperidin-3-yl, 1-acetyl-piperidin-4-yl, tetrahydro-2H-pyran-4-yl, tetrahydro-2H-thiopyran-4-yl, 1,1-dioxo-hexahydro-thiopyran-4-yl, 3-hydroxycyclobutyl, 4-hydroxycyclohexyl, 4-oxocyclohexyl and 4-hydroxy-4-methylcyclohexyl.

In a further aspect, the invention relates to compounds of formula, wherein R² is unsubstituted phenyl or phenyl substituted by one, two or three groups independently selected from the group consisting of C₁₋₇-alkyl, halogen, halogen-C₁₋₇-alkyl, halogen-C₁₋₇-alkoxy and C₁₋₇-alkylsulfonyl. In particular, the invention relates to compounds of formula I, wherein R² is 2-methylphenyl.

Furthermore, compounds of formula I according to the invention are in particular those, wherein R³ and R⁷ are hydrogen.

Compounds of formula I according to the present invention are further those, wherein R⁵ is selected from the group consisting of

halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkylsulfonyl, C₁₋₇-alkylsulfonyloxy,
C₁₋₇-alkylsulfonylamino, C₃₋₇-cycloalkylsulfonylamino,
aminosulfonyl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₇-alkyl-amino, hydroxy-C₁₋₇-alkyl-C₁₋₇-alkyl-amino, an amino acid attached through the amino group of the amino acid,
C₃₋₇-cycloalkyl-amino, wherein C₃₋₇-cycloalkyl is unsubstituted or substituted by hydroxy, hydroxy-C₁₋₇-alkyl or carboxyl,
carboxyl-C₁₋₇-alkyl-aminocarbonyl, carboxyl-C₁₋₇-alkyl-(C₁₋₇-alkyl)-aminocarbonyl,
C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl, hydroxy-C₁₋₇-alkyl-C₁₋₇-alkyl-aminocarbonyl, halogen-hydroxy-C₁₋₇-alkyl-aminocarbonyl,
C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl-C₁₋₇-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₃₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl,
heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahydropyranyl, carboxyl, carboxyl-C₁₋₇-alkyl, C₁₋₇-alkoxy-C₁₋₇-alkyl or C₁₋₇alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl, C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl,
tetrazolyl-C₁₋₇-alkyl-aminocarbonyl and carboxyl-C₁₋₇-alkyl-aminocarbonyl;
and R⁴ and R⁶ are hydrogen.

In particular, the invention relates to compounds of formula I, wherein R⁵ is selected from the group consisting of

halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
C₁₋₇-alkylsulfonyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, and phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl,
C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl,
tetrazolyl-C₁₋₇-alkyl-aminocarbonyl and carboxyl-C₁₋₇-alkyl-aminocarbonyl;
and R⁴ and R⁶ are hydrogen.

More particularly, compounds of formula I according to the in, wherein R⁵ is selected from the group consisting of

halogen, halogen-C₁₋₇-alkyl,
carboxyl, carboxyl-C₁₋₇-alkyl, carboxyl-C₃₋₇-alkenyl, carboxyl-C₁₋₇-alkynyl, C₁₋₇-alkylsulfonyl,
heterocyclyl, said heterocyclyl being unsubstituted or substituted by carboxyl or C₁₋₇-alkylsulfonyl, and
phenyl, said phenyl being unsubstituted or substituted by carboxyl;
and R⁴ and R⁶ are hydrogen.

Even more particularly, R⁵ is C₁₋₇-alkylsulfonyl or heterocyclyl, said heterocyclyl being unsubstituted or substituted by carboxyl or C₁₋₇-alkylsulfonyl, and R⁴ and R⁵ are hydrogen.

The invention also relates to compounds of formula I, wherein R⁴, R⁵ and R⁶ are hydrogen.

In a further aspect, the invention relates to compounds of formula I according to the present invention, wherein R⁶ is selected from the group consisting of

halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkylsulfonyl, C₁₋₇-alkylsulfonyloxy,
C₁₋₇-alkylsulfonylaznino, C₃₋₇-cycloalkylsulfonylamino,
aminosulfonayl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₄-alkyl-amino,
hydroxy-C₁₋₇-alkyl-C₁₋₇-alkyl-amino, an amino acid attached through the amino group of the amino acid,
C₃₋₇-cycloalkyl-amino, wherein C₃₋₇-cycloalkyl is unsubstituted or substituted by hydroxy, hydroxy-C₁₋₄-alkyl or carboxyl,
carboxyl-C₁₋₇-alkyl-aminocarbonyl, carboxyl-C₁₋₇-alkyl-(C₁₋₇-alkyl)-aminocarbonyl,
C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl,
hydroxy-C₁₋₇-alkyl-C₁₋₇-alkyl-aminocarbonyl, halogen-hydroxy-C₁₋₇-alkyl-aminocarbonyl, C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl-C₁₋₇-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₃₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahydropyranyl, carboxyl, carboxyl-C₁₋₇-alkyl, C₁₋₇-alkoxy-C₁₋₇-alkyl or C₁₋₇alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₃₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl, C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl, tetrazolyl-C₁₋₇-alkyl-aminocarbonyl and carboxyl-C₁₋₇-alkyl-aminocarbonyl;
and R⁴ and R⁵ are hydrogen.

Particular compounds of formula I are the following:
5-((R,E)-1-(hydroxyimino)-3-(4-(methylsulfonyl)phenyl)-3-o-tolylpropyl)-1-methylpiperidin-2-one,
5-((R,Z)-1-(hydroxyimino)-3-(4-(methylsulfonyl)phenyl)-3-o-tolylpropyl)-1-methylpiperidin-2-one,
5-[(R)-1-[(E)-hydroxyimino]-3-(4-methanesulfonyl-phenyl)-3-o-tolyl-propyl]-1-methyl-piperidin-2-one,
1-(4-((1R,E)-3-(hydroxyimino)-3-(1-methyl-6-oxopiperidin-3-yl)-1-O-tolylpropyl)phenyl)piperidine-4-carboxylic acid,
sodium 1-(4-((1R,E)-3-(hydroxyimino)-3-(1-methyl-6-oxopiperidin-3-yl)-1-O-tolylpropyl)phenyl)piperidine-4-carboxylate,
5-((R,E)-1-(hydroxyimino)-3-phenyl-3-o-tolylpropyl)-1-methylpiperidin-2-one,
4′-((1R,E)-3-(hydroxyimino)-3-(1-methyl-6-oxopiperidin-3-yl)-1-o-tolylpropyl)biphenyl-4-carboxylic acid,
(E)-1-(4-(3-(4-bromophenyl)-1-(hydroxyimino)-3-o-tolylpropyl)piperidin-1-yl)ethanone,
(E)-3-(4-bromophenyl)-1-(tetrahydro-2H-pyran-4-yl)-3-o-tolylpropan-1-one oxime,
(E)-3-(4-(methylsulfonyl)phenyl)-3-phenyl-1-(tetrahydro-2H-pyran-4-yl)propan-1-one oxime,
(E)-3-(4-bromophenyl)-1-(tetrahydro-2H-thiopyran-4-yl)-3-o-tolylpropan-1-one oxime,
3-(4-Bromo-phenyl)-1-(1,1-dioxo-hexahydro-thiopyran-4-yl)-3-o-tolyl-propan-1-one oxime,
(E)-3-(4-bromophenyl)-1-((1r,4r)-4-hydroxycyclohexyl)-3-o-tolylpropan-1-one oxime,
(E)-4-(3-(4-bromophenyl)-1-(hydroxyimino)-3-o-tolylpropyl)cyclohexanone,
(E)-1-(4-hydroxycyclohexyl)-3-(4-(methylsulfonyl)phenyl)-3-o-tolylpropan-1-one oxime,
(Z)-1-(4-hydroxycyclohexyl)-3-(4-(methylsulfonyl)phenyl)-3-o-tolylpropan-1-one oxime,
(E)-1-((1r,4r)-4-hydroxy-4-methylcyclohexyl)-3-(4-(methylsulfonyl)phenyl)-3-o-tolylpropan-1-one oxime,
(E)-3-(4-bromophenyl)-1-(3-hydroxycyclobutyl)-3-o-tolylpropan-1-one oxime,
   or pharmaceutically acceptable salts thereof.

More particularly, the invention relates to the following compounds of formula I:
5-[(R)-1-[(E)-hydroxyimino]-3-(4-methanesulfonyl-phenyl)-3-o-tolyl-propyl]-1-methyl-piperidin-2-one,
1-(4-((1R,E)-3-(hydroxyimino)-3-(1-methyl-6-oxopiperidin-3-yl)-1-o-tolylpropyl)phenyl)piperidine-4-carboxylic acid,
   or pharmaceutically acceptable salts thereof.

The pharmaceutically acceptable salts of the compounds of formula I also individually constitute compounds of the present invention of particular interest.

An example thereof is the following:
sodium 1-(4-((1R,E)-3-(hydroxyimino)-3-(1-methyl-6-oxopiperidin-3-yl)-1-o-tolylpropyl)phenyl)piperidine-4-carboxylate.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbens or eluant). The invention embraces all of these forms.

In particular, the compounds of formula I of the present invention are oximes and thus can exist in two isomeric forms at the C═N—OH double bond, i.e. the E- (or anti) and the Z- (or syn) isomer.

It will be appreciated, that the compounds of general formula I in this invention may be derivatised at functional groups to provide derivatives which are capable of conversion back to the parent compound in vivo. Physiologically acceptable and metabolically labile derivatives, which are capable of producing the parent compounds of general formula I in vivo are also within the scope of this invention.

A further aspect of the present invention is the process for the manufacture of compounds of formula I as defined above, which process comprises reacting a ketone of the formula II

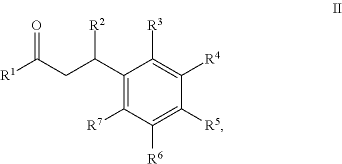

wherein R¹ to R⁷ are as defined above, with hydroxylamine hydrochloride in the presence of a base to obtain a compound of the formula I

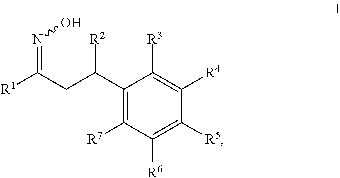

wherein R¹ to R⁷ are as defined above, and, if desired,
converting the compound obtained into a pharmaceutically acceptable salt.

Appropriate bases are for example are for example sodium hydroxide, sodium hydrogen carbonate or sodium acetate. The reaction is carried out in a suitable solvent such as for example ethanol, methanol, water, or mixtures thereof; at temperatures between room temperature and 150° C., optionally under microwave irradiation.

Optionally, the ratio of E and Z isomers of the compound of formula I can be modified by treating the obtained compound of formula I with acids such as hydrochloric acid in solvents such as ethanol, 1,2-dimethoxyethane and dioxane or in mixtures thereof at temperatures between room temperature and reflux of the solvent. The E and Z isomers can be separated by column chromatography or by HPLC.

The invention further relates to compounds of formula I as defined above obtainable according to a process as defined above.

In detail, the ketones of formula II can be prepared as described below in schemes 1-6, or in analogy to the methods described below with methods known in the art. All starting materials are either commercially available, described in the literature or can be prepared by methods well known in the art or by methods in analogy to those described below.

Compounds of general formula II can be produced as outlined in scheme 2. R^{a} is lower alkyl, e.g. methyl or ethyl, R^{b} is lower alkyl, e.g. methyl, ethyl, or isopropyl.

In step a, scheme 1, ester 1 is reacted with dialkyl methyl phosphonate 2 in the presence of 2.1 equivalents of a suitable base, leading to β-ketophosphonate 3. The reaction is performed as described in the literature (J. Org. Chem. 2009, 74, 7574) in a suitable solvent, e.g. tetrahydrofuran, at temperatures around 0° C. In particular, the base is lithium diisopropylamide.

In step b, scheme 1, β-ketophosphonate 3 undergoes a Horner-Wadsworth-Emmons reaction with aldehyde 4, leading to enone 5, using conditions and reagents described in the art. Particularly, the reaction is performed in the presence of a base, e.g. potassium carbonate, triethylamine, or 1,8-diazabicycloundec-7-ene, in a solvent such as tetrahydrofuran or ethanol, at temperatures between −20° C. and the boiling point of the solvent.

In step c, scheme 1, ketone II is obtained from enone 5 by a 1,4-addition with a suitable reagent, as described in the literature. For instance, enone 5 is reacted with a Grignard reagent, R²—Mg—X (X═Cl, Br, I), optionally in the presence of catalytic amounts of copper(I) iodide, in a solvent such as tetrahydrofuran, at temperatures between −78° C. and +20° C. Alternatively, in the case where R² is aryl or heteroaryl, enone 5 may be reacted with a boronic acid, R²B(OH)₂, in the presence of a palladium catalyst system, e.g. palladium(II) acetate/triphenylphosphine, and a base, e.g. cesium carbonate, in toluene/chloroform, at temperatures between 60° C. and 110° C.

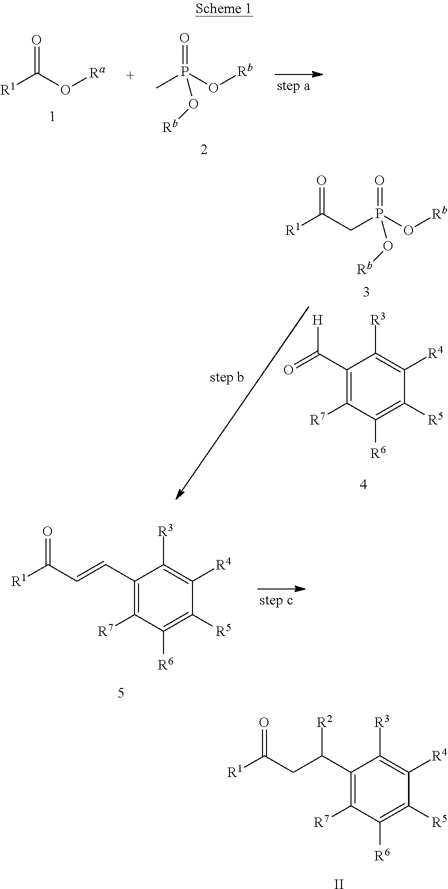

In scheme 1, R¹, R², R³, R⁴, R⁵, R⁶, and R⁷ are as defined above.

Compounds of formula II may also be produced as outlined in scheme 2. Thus, N-methoxy-N-methylamide 6 is reacted with an organolithium or organomagnesium reagent, R¹—Li or R¹—MgX (X═Cl, Br, I), at temperatures between −100° C. and −70° C., in a solvent such as tetrahydrofuran or 2-methyltetrahydrofuran. The required organometallic reagent is generated from the appropriately substituted halide using methods well known in the art. For instance, the organomagnesium reagent is produced by reaction of the corresponding cycloalkyl halide or heterocyclyl halide, R¹—X (X═Cl, Br, I) with magnesium in a solvent such as tetrahydrofuran or diethyl ether, optionally in the presence of catalytic amounts of radical initiators such as iodine, iodotrimethylsilane, and/or 1,2-dichloroethane, at temperatures between 0° C. and the boiling point of the solvent. Alternatively, the organomagnesium reagent may be produced from the halide R¹—X (X═Cl, Br, I) by reaction with isopropylmagnesium chloride, in a solvent such as tetrahydrofuran, at temperatures between −20° C. and +20° C.

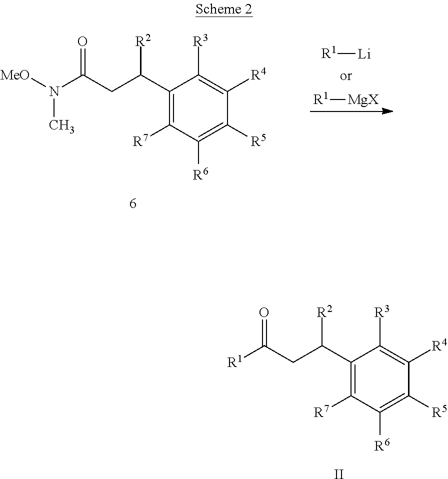

In scheme 2, R¹, R², R³, R⁴, R⁵, R⁶, and R⁷ are as defined before.

Compounds of formula II in which R¹ is 2-oxopiperidinyl are represented by the general formula IIa:

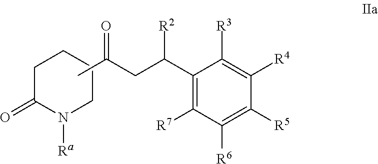

In formula IIa, R^{a} is hydrogen or C₁₋₇-alkyl, R², R³, R⁴, R⁵, R⁶, and R⁷ are as defined before.

Compounds of formula IIa can also be produced as outlined in scheme 3. In scheme 3, R^{a} is hydrogen or C₁₋₇-alkyl.

In step a, scheme 3, N-methoxy-N-methylamide 6 is reacted with the organolithium or organomagnesium derivative of methoxypyridine 7 (M=Li or MgX, with X═Cl, Br, I), leading to ketone 8. This reaction is performed at temperatures between −100° C. and −70° C., in a solvent such as tetrahydrofuran or 2-methyltetrahydrofuran. The appropriate organometallic pyridine reagent is generated from the appropriately substituted bromo-2-methoxypyridine using methods well known in the art. For instance, the organolithium reagent is produced by reaction with n-butyllithium in a solvent such as tetrahydrofuran or 2-methyltetrahydrofuran, at temperatures between −100° C. and −70° C.

In step b, scheme 3, the methyl group of the 2-methoxypyridine subunit of 8 is cleaved, leading to 2-pyridone 9. This reaction is performed in the presence of an acid, e.g., hydrochloric acid, in solvents such as water, tetrahydrofuran, 1,4-dioxane, or mixtures thereof, at temperatures between 20° C. and 100° C.

In optional step c, scheme 3, the nitrogen of the 2-pyridone subunit of 9 is alkylated, leading to compound 10. This reaction is performed using methods and reagents known in the art, e.g. using alkyl halide R^{a}—X (X═Cl, Br, I), in the presence of a base, e.g. potassium carbonate, sodium hydride, or sodium hydroxide, in a solvent such as N,N-dimethylformamide, N,N-dimethylacetamide, tetrahydrofuran, or ethanol.

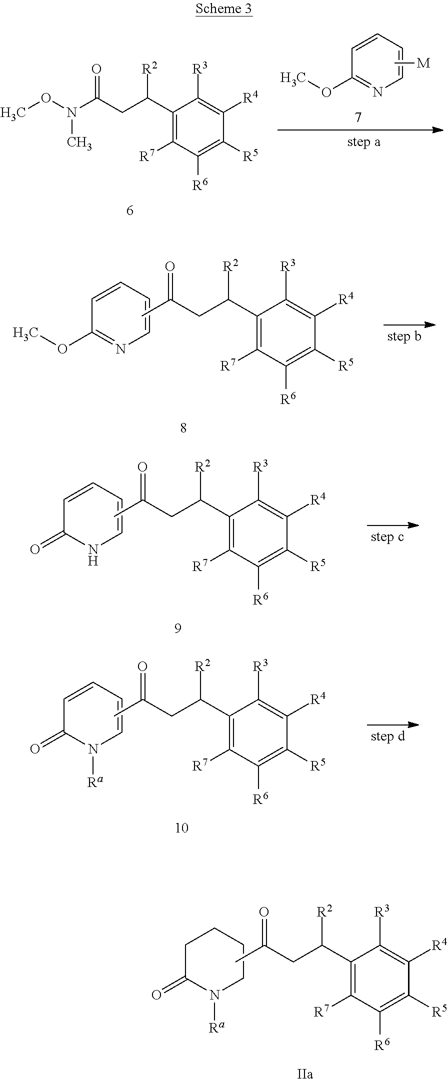

In scheme 3, R², R³, R⁴, R⁵, R⁶, and R⁷ are as defined before.

In step d, scheme 3,2-pyridone 10 is transformed to the piperidin-2-one IIa by hydrogenation of the heteroaromatic ring. This reaction is performed using hydrogen gas at pressures between 1 bar and 100 bar, in a solvent such as ethanol, methanol, or acetic acid, in the presence of a suitable catalyst such as palladium on activated charcoal or platinum(IV) oxide, at temperatures between 0° C. and 100° C. In this reaction the concomitant reduction of the ketone group of 10 to the corresponding secondary alcohol may occur. In such a case, the secondary alcohol may be re-oxidized to the ketone using a suitable reagent, e.g., 1,1,1-triacetoxy-1,1-dihydro-1,2-benziodoxol-3(1H)-one in dichloromethane at room temperature. Alternatively, the ketone 10 may be regenerated from the secondary alcohol intermediate using sodium hypochlorite, in a two-phase mixture of water and dichloromethane, in the presence of sodium hydrogencarbonate and catalytic amounts of sodium bromide or potassium bromide and 2,2,6,6-tetramethylpiperidin-1-oxyl radical, at temperatures between 0° C. and 25° C.

N-Methoxy-N-methylamides of general formula 6 can be produced as outlined in scheme 4. R^{a} is lower alkyl, e.g. methyl or ethyl.

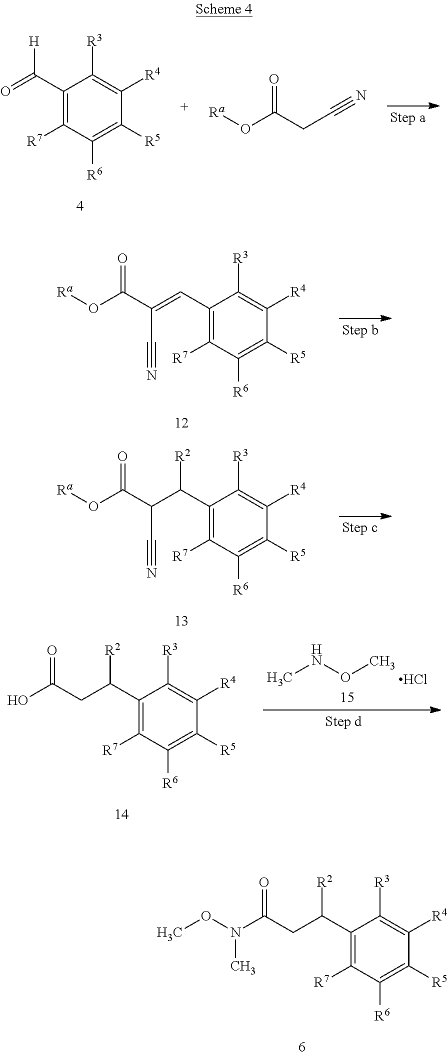

In scheme 4, R², R³, R⁴, R⁵, R⁶, and R⁷ are as defined before.

In step a, scheme 4, aldehyde 4 is condensed with alkyl cyanoacetate 11, leading to 12. The reaction is performed in the presence of a base, e.g. potassium carbonate, potassium hydroxide, or piperidine, at temperatures between 20° C. and 120° C., in solvents such as ethanol, toluene or acetic acid.

In step b, scheme 4, α,β-unsaturated cyanoester 12 undergoes a 1,4-addition reaction with an appropriate organomagnesium halide reagent, R²—MgX (X═Cl, Br), leading to 13. This reaction is performed in a solvent such as toluene or tetrahydrofuran, at temperatures between 0° C. and 110° C.

In step c, scheme 4, cyanoester 13 undergoes hydrolysis and decarboxylation, leading to carboxylic acid 14. This reaction is performed using methods and reagents known in the art, e.g. using acids such as acetic acid, sulfuric acid, hydrochloric acid or mixtures thereof, at temperatures between 60° C. and 120° C.

Carboxylic acid intermediate 14 containing an asymmetric carbon atom may be separated into its enantiomers using methods known in the art, e.g., by fractional crystallization using an optically pure base, e.g., 1-phenylethylamine, or by chromatography using a chiral stationary phase.

In step d, scheme 4, carboxylic acid is converted to the N-methoxy-N-methylamide 6 using methods and reagents known in the art. For instance, the reaction is carried out using commercially available N,O-dimethylhydroxylamine hydrochloride (15) in the presence of a coupling agent such as 1,1′-carbonyldiimidazole, N,N′-dicyclohexylcarbodiimide, 1-(3-dimethylaminopropyl)-3-ethyl-carbodiimide hydrochloride, O-(benzotriazol-1-yl)-N,N,N′,N′-tetramethyluronium hexafluoro-phosphate, O-(7-azabenzotriazol-1-yl)-N,N,N′,N′-tetramethyluronium hexafluoro-phosphate or bromo-tris-pyrrolidino-phosphonium hexafluorophosphate, in aprotic solvents such as dichloromethane, tetrahydrofuran, N,N-dimethylformamide, N-methylpyrrolidinone and mixtures thereof at temperatures between −40° C. and 80° C. in the presence or absence of a base such as triethylamine, diisopropylethylamine, 4-methylmorpholine and/or 4-(dimethylamino)pyridine. Alternatively, this reaction can be performed in two steps involving first formation of the acyl halide derivative of 14 and subsequent coupling reaction with 15 in the presence of a base. Typically employed reagents for the formation of the acyl chloride are thionyl chloride, phosphorus pentachloride, oxalyl chloride or cyanuric chloride, and the reaction is generally conducted in the absence of a solvent or in the presence of an aprotic solvent like dichloromethane, toluene or acetone. A base can optionally be added, like for example pyridine, triethylamine, diisopropylethylamine or 4-methylmorpholine, and catalytic amounts of N,N-dimethylformamide may be used. The obtained acyl chloride can be isolated or reacted as such with 15 in an aprotic solvent, like dichloromethane, tetrahydrofuran or acetone, in the presence of a base. Typical bases are triethylamine, 4-methylmorpholine, pyridine, diisopropylethylamine or 4-(dimethylamino)pyridine or mixtures thereof.

N-Methoxy-N-methylamide derivative 6 containing an asymmetric carbon may be separated into its enantiomer using methods known in the art, e.g. chromatography using a chiral stationary phase or a chiral eluent.

Compounds of formula II, in which one of R⁴, R⁵ or R⁶ is Br are represented by the general formula IIb.

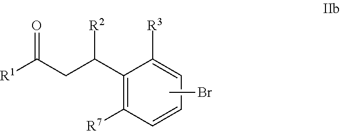

Compounds of general formula IIb can further be elaborated to ketone intermediates IIc, IId, or IIe using methods described in the literature, e.g. as outlined in scheme 5.

For instance, for the introduction of an amine moiety Buchwald-Hartwig conditions can be used. Therefore the bromoketone IIb is reacted with a primary or secondary amine (16), leading to arylamine IIc. This reaction is performed in the presence of a catalyst system containing a palladium source such as tris(dibenzylidene-acetone)dipalladium(0) and a ligand such as 2-(di-tert-butylphosphino)biphenyl or 2-dicyclohexylphosphino-2′,4′,6′-triisopropylbiphenyl in the presence of a base such as sodium tert-butylate, in a solvent such as toluene or 1,4-dioxane, at temperatures between 20° C. and 110° C. (step a, scheme 5).

As shown in step b, scheme 5, bromoketone IIb can be transformed into the corresponding alkyl aryl sulfone lid by reaction with the sodium alkanesulfinate salt 17 (R^{E}=C₁₋₇ alkyl). This reaction is performed using methods and reagents known in the art, e.g. in the presence of copper(I) iodide and proline sodium salt, in a solvent such as dimethyl sulfoxide, at temperatures between 100° C. and 150° C.

Suzuki reaction of bromoketone IIb with a suitably substituted boronic acid 18 (R^{F}=substituted aryl, heteroaryl, alkenyl, alkyl) or equivalent organoboron reagent leads to compound IIe (step c, scheme 5). This reaction is performed in the presence of a suitable catalyst, preferably a palladium catalyst such as dichloro[1,1′-bis(diphenylphosphino)-ferrocene]palladium(II) dichloromethane adduct or tetrakis(triphenylphosphine)palladium(0) and a base, particularly sodium carbonate, sodium hydrogencarbonate, potassium fluoride, potassium carbonate, or triethylamine in solvents such as dioxane, water, toluene, N,N-dimethylformamide or mixtures thereof.

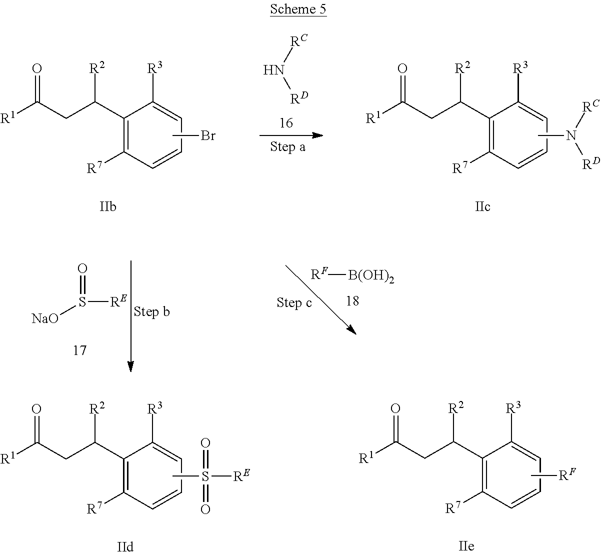

In scheme 5, R¹, R², R³, R⁷, and R¹⁰ are as defined before.

Intermediates of formula 8, 9 or 10 in which one of R⁴, R⁵ or R⁶ is Br are represented by the general formula 19. R¹⁰ corresponds to 2-methoxypyridyl or 2-oxo-dihydropyridyl with an optional C₁₋₇-alkyl substituent at the ring nitrogen atom.

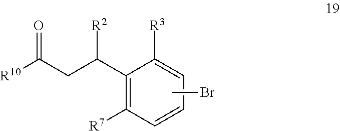

In a similar manner to the transformations shown in scheme 5, compounds of general formula 19 can further be elaborated to ketone intermediates 20, 21, or 22 using methods described in the literature, e.g. as outlined in scheme 6.

For instance, arylamine 20 can be obtained from bromoketone 19 and amine 16 in analogy to scheme 5, step a. Similarly, alkyl aryl sulfone 21 may be produced from bromoketone 19 and sodium alkanesulfinate 17 in analogy to scheme 5, step b. Furthermore, compound 22 can be synthesised from bromoketone 19 and boronic acid 18 in analogy to scheme 5, step c.

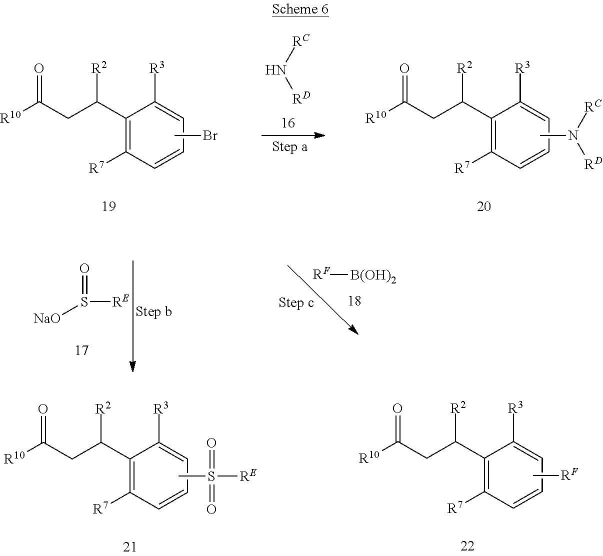

In scheme 6, R², R³, and R⁷ are as defined before.

Functional groups that are incompatible with the reaction conditions described in schemes 1-6 can be protected using methods and reagents known in the art, e.g., as described in P. J. Kocienski, “Protecting groups”, Georg Thieme Verlag, 3rd ed., 2004.

As described herein before, the compounds of formula I of the present invention can be used as medicaments for the treatment of diseases which are associated with the modulation of GPBAR1 activity.

As compounds of formula I of the invention are agonists of the GPBAR1 receptor, the compounds will be useful for lowering glucose, lipids, and insulin resistance in diabetic patients and in non-diabetic patients who have impaired glucose tolerance or who are in a pre-diabetic condition. The compounds of formula I are further useful to ameliorate hyperinsulinemia, which often occurs in diabetic or pre-diabetic patients, by modulating the swings in the level of serum glucose that often occurs in these patients. The compounds of formula I are also useful in reducing the risks associated with metabolic syndrome, in reducing the risk of developing atherosclerosis or delaying the onset of atherosclerosis, and reducing the risk of angina, claudication, heart attack, stroke, and coronary artery disease. By keeping hyperglycemia under control, the compounds are useful to delay or for preventing vascular restenosis and diabetic retinopathy.

The compounds of formula I of the present invention are useful in improving or restoring β-cell function, so that they may be useful in treating type 1 diabetes or in delaying or preventing a patient with type 2 diabetes from needing insulin therapy. The compounds may be useful for reducing appetite and body weight in obese subjects and may therefore be useful in reducing the risk of co-morbidities associated with obesity such as hypertension, atherosclerosis, diabetes, and dyslipidemia. By elevating the levels of active GLP-1 in vivo, the compounds are useful in treating neurological disorders such as Alzheimer's disease, multiple sclerosis, and schizophrenia.

Thus, the expression “diseases which are associated with the modulation of GPBAR1 activity” means diseases such as metabolic, cardiovascular, and inflammatory diseases, for example diabetes, particularly type 2 diabetes, gestational diabetes, impaired fasting glucose, impaired glucose tolerance, insulin resistance, hyperglycemia, obesity, metabolic syndrome, ischemia, myocardial infarction, retinopathy, vascular restenosis, hypercholesterolemia, hypertriglyceridemia, dyslipidemia or hyperlipidemia, lipid disorders such as low HDL cholesterol or high LDL cholesterol, high blood pressure, angina pectoris, coronary artery disease, atherosclerosis, cardiac hypertrophy, rheumatoid arthritis, asthma, chronic obstructive pulmonary disease (COPD), psoriasis, ulcerative colitis, Crohn's disease, disorders associated with parenteral nutrition especially during small bowel syndrome, irritable bowel syndrome (IBS), allergy diseases, fatty liver (e.g. non-alcoholic fatty liver disease, NAFLD), liver fibrosis (e.g. non-alcoholic steatohepatitis, NASH), primary sclerosing cholangitis (PSC), liver cirrhosis, primary biliary cirrhosis (PBC), liver colestasis, kidney fibrosis, anorexia nervosa, bulimia nervosa and neurological disorders such as Alzheimer's disease, multiple sclerosis, schizophrenia and impaired cognition.

In a particular aspect, the expression “diseases which are associated with the modulation of GPBAR1 activity” relates to diabetes, particularly type 2 diabetes, gestational diabetes, impaired fasting glucose, impaired glucose tolerance, hyperglycemia, metabolic syndrome, obesity, hypercholesterolemia and dyslipidemia.

The invention also relates to pharmaceutical compositions comprising a compound as defined above and a pharmaceutically acceptable carrier and/or adjuvant. More specifically, the invention relates to pharmaceutical compositions useful for the treatment of diseases which are associated with the modulation of GPBAR1 activity.

Further, the invention relates to compounds of formula I as defined above for use as therapeutically active substances, particularly as therapeutically active substances for the treatment of diseases which are associated with the modulation of GPBAR1 activity. In particular, the invention relates to compounds of formula I for use in diabetes, particularly type 2 diabetes, gestational diabetes, impaired fasting glucose, impaired glucose tolerance, hyperglycemia, metabolic syndrome, obesity, hypercholesterolemia and dyslipidemia, more particularly for use in diabetes, preferably type 2 diabetes, gestational diabetes or hyperglycemia.

In another aspect, the invention relates to a method for the treatment a of diseases which are associated with the modulation of GPBAR1 activity, which method comprises administering a therapeutically active amount of a compound of formula I to a human being or animal. In particular, the invention relates to a method for the treatment of diabetes, particularly type 2 diabetes, gestational diabetes, impaired fasting glucose, impaired glucose tolerance, hyperglycemia, metabolic syndrome, obesity, hypercholesterolemia and dyslipidemia, more particularly for the treatment of diabetes, preferably type 2 diabetes, gestational diabetes or hyperglycemia.

The invention further relates to the use of compounds of formula I as defined above for the treatment of diseases which are associated with the modulation of GPBAR1 activity.

In addition, the invention relates to the use of compounds of formula I as defined above for the preparation of medicaments for the treatment of diseases which are associated with the modulation of GPBAR1 activity. In particular, the invention relates to the use of compounds of formula I as defined above for the preparation of medicaments for the treatment of diabetes, particularly type 2 diabetes, gestational diabetes, impaired fasting glucose, impaired glucose tolerance, hyperglycemia, metabolic syndrome, obesity, hypercholesterolemia and dyslipidemia, more particularly for the preparation of medicaments for the treatment of diabetes, preferably type 2 diabetes, gestational diabetes or hyperglycemia.

Also contemplated herein is a combination therapy using one or more compounds of formula I or compositions of the present invention, or a pharmaceutically acceptable salts thereof, in combination with one or more other pharmaceutically active compounds independently selected from the group consisting of the following:

(a) human peroxisome proliferator activated receptor (PPAR) gamma agonists (e.g., thiazolidinediones and glitazones, e.g., rosiglitazone, troglitazone, pioglitazone, englitazone, balaglitazone, and netoglitazone),
(b) biguanides such as metformin, metformin hydrochloride, buformin and phenformin,
(c) dipeptidyl peptidase IV (DPP-4) inhibitors, such as sitagliptin, sitagliptin phosphate, saxagliptin, vildagliptin, alogliptin, carmegliptin, and denagliptin,
(d) incretins such as glucagon-like peptide-1 (GLP-1) receptor agonists such as exenatide (Byetta™), liraglutide (Victoza™), GLP-1(7-36) amide and its analogs, GLP-1(7-37) and its analogs, AVE-0010 (ZP-10), R1583 (taspoglutide), GSK-716155 (albiglutide, GSK/Human Genome Sciences), BRX-0585 (Pfizer/Biorexis) and CJC-1134-PC (Exendin-4:PC-DAC™) or glucose-dependent insulinotropic peptide (GIP),
(e) insulin or insulin analogs such as LysPro insulin or inhaled formulations comprising insulin,
(f) sulfonylureas such as tolazamide, chlorpropamide, glipizide, glimepiride, glyburide, glibenclamide, tolbutamide, acetohexamide or glypizide,
(g) α-glucosidase inhibitors such as miglitol, acarbose, epalrestat, or voglibose,
(h) cholesterol biosynthesis inhibitors such as HMG CoA reductase inhibitors, e.g., lovastatin, simvastatin, pravastatin, fluvastatin, atorvastatin, rosuvastatin, cerivastatin, itavastin, nisvastatin and rivastatin, or squalene epoxidase inhibitors, e.g., terbinafine,
(i) plasma HDL-raising agents such as CETP inhibitors e.g., anacetrapib, torcetrapib and dalcetrapib, or PPAR alpha agonists, e.g., gemfibrozil, clofibrate, fenofibrate and bezafibrate,
(j) PPAR dual alpha/gamma agonists such as muraglitazar, naveglitazar, aleglitazar, tesaglitazar, peliglitazar, farglitazar and JT-501,
(k) bile acid sequestrants, e.g., anion exchange resins, or quaternary amines (e.g., cholestyramine or colestipol)), or ileal bile acid transporter inhibitors (BATi);
(l) nicotinyl alcohol, nicotinic acid, niacinamide or salts thereof,
(m) cholesterol absorption inhibitors such as ezetimibe or acyl-Coenzyme A:cholesterol O-acyl transferase (ACAT) inhibitors such as avasimibe,
(n) selective estrogen receptor modulators such as raloxifene or tamoxifen) or LXR alpha or beta agonists, antagonists or partial agonists (e.g., 22(R)-hydroxycholesterol, 24(S)-hydroxycholesterol, T0901317 or GW3965);
(o) microsomal triglyceride transfer protein (MTP) inhibitors, alpha2-antagonists and imidazolines (e.g., midaglizole, isaglidole, deriglidole, idazoxan, efaroxan, fluparoxan),
(p) insulin secretagogues such as linogliride, nateglinide, repaglinide, mitiglinide calcium hydrate or meglitinide);
(q) SGLT-2 inhibitors (e.g., dapagliflozin, sergliflozin and tofoglifozin),
(s) glucokinase activators such as the compounds disclosed in e.g., WO 00/58293 A1;
(t) protein tyrosine phosphatase-1B (PTP-1B) inhibitors,
(u) glucagon receptor antagonists,
(v) anti-obesity agents such as fenfluramine, dexfenfluramine, phentiramine, sibutramine, orlistat, neuropeptide Y1 or Y5 antagonists, neuropeptide Y2 agonists, MC4R (melanocortin 4 receptor) agonists, cannabinoid receptor 1 (CB-1) antagonists/inverse agonists, and β3 adrenergic receptor agonists (e.g., GW-320659), nerve growth factor agonist (e.g., axokine), growth hormone agonists (e.g., AOD-9604), 5-HT (serotonin) reuptake/transporter inhibitors (e.g., Prozac), DA (dopamine) reuptake inhibitors (e.g., Buproprion), 5-HT, NA and DA reuptake blockers, steroidal plant extracts (e.g., P57), CCK-A (cholecystokinin-A) agonists, GHSR1a (growth hormone secretagogue receptor) antagonist/inverse agonists, ghrelin antibody, MCH1R (melanin concentrating hormone 1R) antagonists (e.g., SNAP 7941), MCH2R (melanin concentrating hormone 2R) agonist/antagonists, H3 (histamine receptor 3) inverse agonists or antagonists, HI (histamine 1 receptor) agonists, FAS (fatty acid synthase) inhibitors, ACC-2 (acetyl-CoA carboxylase-1) inhibitors, DGAT-2 (diacylglycerol acyltransferase 2) inhibitors, DGAT-1 (diacylglycerol acyltransferase 1) inhibitors, CRF (corticotropin releasing factor) agonists, Galanin antagonists, UCP-1 (uncoupling protein-1), 2 or 3 activators, leptin or a leptin derivatives, opioid antagonists, orexin antagonists, BRS3 agonists, IL-6 agonists, a-MSH agonists, AgRP antagonists, BRS3 (bombesin receptor subtype 3) agonists, 5-HT1B agonists, POMC antagonists, CNTF (ciliary neurotrophic factor or CNTF derivative), Topiramate, glucocorticoid antagonist, 5-HT_{2C} (serotonin receptor 2C) agonists (e.g., Lorcaserin), PDE (phosphodiesterase) inhibitors, fatty acid transporter inhibitors, dicarboxylate transporter inhibitors, glucose transporter inhibitors,
(w) anti-inflammatory agents such as cyclooxygenase-2 (COX-2) inhibitors (e.g., rofecoxib and celecoxib); glucocorticoids, azulfidine, thrombin inhibitors (e.g., heparin, argatroban, melagatran, dabigatran) and platelet aggregation inhibitors (e.g., glycoprotein Ilb/IIIa fibrinogen receptor antagonists or aspirin), and ursodeoxycholic acid (UDCA) and norursodeoxycholic acid (norUDCA) and
(y) antihypertensives such as beta blockers (e.g., angiotensin II receptor antagonists such as losartan, eprosartan, irbesartan, tasosartan, telmisartan or valsartan; angiotensin converting enzyme inhibitors such as enalapril, captopril, cilazapril, ramapril, zofenopril, lisinopril and fosinopril; calcium channel blockers such as nifedipine and diltiazam and endothelian antagonists.

Such other pharmaceutically active compounds may be administered in an amount commonly used therefore, contemporaneously or sequentially with a compound of the formula I or a pharmaceutically acceptable salt thereof. In the treatment of patients who have type 2 diabetes, insulin resistance, obesity, metabolic syndrome, neurological disorders, and co-morbidities that accompany these diseases, more than one pharmaceutically active compound is commonly administered. The compounds of formula I of this invention may generally be administered to a patient who is already taking one or more other drugs for these conditions. When a compound of formula I is used contemporaneously with one or more other pharmaceutically active compounds, a pharmaceutical composition in an unit dosage form containing such other pharmaceutically active compounds and the compound of the formula I is preferred. Thus, the invention also relates to a pharmaceutical composition containing a compound of formula I in combination with one or more other pharmaceutically active compounds as defined above. When used in combination with one or more other active ingredients, the compound of formula I of the present invention and the other pharmaceutically active compounds may be used in lower doses than when each is used singly. These kinds of pharmaceutical compositions are also included in the invention.

However, the combination therapy also includes therapies in which the compound of formula I and one or more other pharmaceutically active compounds are administered in different dosage forms, but with overlapping schedules. The invention thus also relates to a method for the treatment a of diseases which are associated with the modulation of GPBAR1 activity, which method comprises administering a therapeutically active amount of a compound of formula I in combination with one or more other pharmaceutically active compounds to a human being or animal.

### Pharmacological Test

The following test was carried out in order to determine the activity of the compounds of formula I:

The cDNA of the human GPBAR1 receptor (Genbank: NM_{—}170699 with the exception of a silent C:G mutation at position 339 from the start codon) was amplified by polymerase chain reaction (PCR) from human cDNA and inserted into pCineo (Promega) by standard methods (Current Protocols in Molecular Biology, Wiley Press, ed. Ausubel et al.). The final clone was verified by DNA sequence analysis. The plasmid was transfected into CHO cells deficient in dihydrofolate reductase activity (CHO-dhfr-) using Lipofectamine plus (Invitrogen). Clones were isolated in limited dilution conditions and identified by activities in the cAMP assay using lithocholic acid as agonist. A clonal cell line displaying the greatest activity in cAMP increases was selected and identified as giving consistently good responses for up to at least 20 passages.

cAMP Assay

CHO-dhfr(minus) cells expressing human GPBAR1 receptors are seeded 17-24 hours prior to the experiment 50.000 cells per well in a black 96 well plate with flat clear bottom (Corning Costar #3904) in DMEM (Invitrogen No. 31331), 1×HT supplement, with 10% fetal calf serum and incubated at 5% CO₂ and 37° C. in a humidified incubator. The growth medium was exchanged with Krebs Ringer Bicarbonate buffer with 1 mM IBMX and incubated at 30° C. for 30 min. Compounds were added to a final assay volume of 100 μl and incubated for 30 min at 30° C. The assay was stopped by the addition of 50 μl lysis reagent (Tris, NaCl, 1.5% Triton X100, 2.5% NP40, 10% NaN₃) and 50 μl detection solutions (20 μM mAb Alexa700-cAMP 1:1, and 48 μM Ruthenium-2-AHA-cAMP) and shaken for 2 h at room temperature. The time-resolved energy transfer is measured by a TRF reader (Evotec Technologies GmbH, Hamburg Germany), equipped with a ND:YAG laser as excitation source. The plate is measured twice with the excitation at 355 nm and at the emission with a delay of 100 ns and a gate of 100 ns, total exposure time 10 s at 730 (bandwidth 30 nm) or 645 nm (bandwidth. 75 nm), respectively. The measured signal at 730 nm has to be corrected for the ruthenium background, the direct excitation of Alexa and the buffer control. The FRET signal is calculated as follows: FRET=T730−Alexa730−P(T645−B645) with P=Ru730−B730/Ru645−B645, where T730 is the test well measured at 730 nM, T645 is the test well measured at 645 nm, B730 and B645 are the buffer controls at 730 nm and 645 nm, respectively. cAMP content is determined from the function of a standard curve spanning from 10 μM to 0.13 nM cAMP.

EC₅₀ values were determined using Activity Base analysis (ID Business Solution, Limited). The EC₅₀ values for a wide range of bile acids generated from this assay were in agreement with the values published in the scientific literature. Specificity for GPBAR1 was tested in non-transfected CHO cells in the same assay as above.

The compounds according to formula I have an activity in the above assay (EC₅₀) preferably of 0.5 nM to 10 μM, more preferably of 0.5 nM to 1 μM and most preferably of 0.5 nM to 100 nM.

For example, the following compounds showed the following human EC₅₀ values in the functional cAMP assay described above:

| | human EC₅₀ |
|---|---|
| Example | [μM] |
| 1 | 0.118 |
| 2 | 8.672 |
| 3 | 0.145 |
| 4 | 0.029 |
| 5 | 0.042 |
| 6 | 0.051 |
| 7 | 0.558 |
| 8 | 8.266 |
| 9 | 0.118 |
| 10 | 0.799 |
| 11 | 9.950 |
| 12 | 9.732 |
| 13 | 1.999 |
| 14 | 0.601 |
| 15 | 0.680 |
| 16 | 7.720 |
| 17 | 9.805 |
| 18 | 3.846 |

### Pharmaceutical Compositions

The compounds of formula I and their pharmaceutically acceptable salts can be used as medicaments, e.g., in the form of pharmaceutical preparations for enteral, parenteral or topical administration. They can be administered, for example, perorally, e.g., in the form of tablets, coated tablets, dragées, hard and soft gelatin capsules, solutions, emulsions or suspensions, rectally, e.g., in the form of suppositories, parenterally, e.g., in the form of injection solutions or suspensions or infusion solutions, or topically, e.g., in the form of ointments, creams or oils. Oral administration is preferred.

The production of the pharmaceutical preparations can be effected in a manner which will be familiar to any person skilled in the art by bringing the described compounds of formula I and their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatin capsules. Suitable carrier materials for soft gelatin capsules are, for example, vegetable oils, waxes, fats and semi-solid and liquid polyols (depending on the nature of the active ingredient no carriers might, however, be required in the case of soft gelatin capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage of the compounds of formula I can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the compound could be administered with one or several daily dosage units, e.g., in 1 to 3 dosage units.

The pharmaceutical preparations conveniently contain about 1-500 mg, preferably 1-100 mg, of a compound of formula I.

The following examples C1 to C5 illustrate typical compositions of the present invention, but serve merely as representative thereof.

### Example C1

Film coated tablets containing the following ingredients can be manufactured in a conventional manner:

| | |
|---|---|
| Ingredients | Per tablet |

| | | |
|---|---|---|
| Kernel: | | |
| Compound of formula I | 10.0 mg | 200.0 mg |
| Microcrystalline cellulose | 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |
| Film Coat: | | |
| Hydroxypropyl methyl cellulose | 3.5 mg | 7.0 mg |
| Polyethylene glycol 6000 | 0.8 mg | 1.6 mg |
| Talc | 1.3 mg | 2.6 mg |
| Iron oxide (yellow) | 0.8 mg | 1.6 mg |
| Titanium dioxide | 0.8 mg | 1.6 mg |

The active ingredient is sieved and mixed with microcrystalline cellulose and the mixture is granulated with a solution of polyvinylpyrrolidone in water. The granulate is mixed with sodium starch glycolate and magesiumstearate and compressed to yield kernels of 120 or 350 mg respectively. The kernels are lacquered with an aqueous solution/suspension of the above mentioned film coat.

### Example C2

Capsules containing the following ingredients can be manufactured in a conventional manner:

| Ingredients | Per capsule |
|---|---|
| Compound of formula I | 25.0 mg |
| Lactose | 150.0 mg |
| Maize starch | 20.0 mg |
| Talc | 5.0 mg |

The components are sieved and mixed and filled into capsules of size 2.

### Example C3

Injection solutions can have the following composition:

| Compound of formula I | 3.0 | mg |
|---|---|---|
| Polyethylene glycol 400 | 150.0 | mg |
| Acetic acid | q.s. ad pH 5.0 | |
| Water for injection solutions | ad 1.0 | ml |

The active ingredient is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example C4

Soft gelatin capsules containing the following ingredients can be manufactured in a conventional manner:

| Capsule contents | | |
|---|---|---|
| Compound of formula I | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 | mg (dry matter) |
| Titanium dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example C5

Sachets containing the following ingredients can be manufactured in a conventional manner:

| | |
|---|---|
| Compound of formula I | 50.0 mg |
| Lactose, fine powder | 1015.0 mg |
| Microcrystalline cellulose (AVICEL PH 102) | 1400.0 mg |
| Sodium carboxymethyl cellulose | 14.0 mg |
| Polyvinylpyrrolidone K 30 | 10.0 mg |
| Magnesiumstearate | 10.0 mg |
| Flavoring additives | 1.0 mg |

The active ingredient is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

The following examples serve to illustrate the present invention in more detail. They are, however, not intended to limit its scope in any manner.

### EXAMPLES

### Abbreviations

CAS RN=Chemical Abstracts registry number, DMF=N,N-dimethylformamide, EI=electron impact, HPLC=high performance liquid chromatography, min=minutes, MS=mass spectrum, sat.=saturated, aq.=aqueous, THF=tetrahydrofuran.

### Examples 1 and 2

### 5-((R,E)-1-(Hydroxyimino)-3-(4-(methylsulfonyl)phenyl)-3-o-tolylpropyl)-1-methylpiperidin-2-one

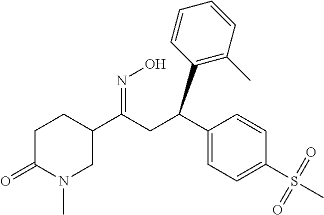 [SMILES: Cc1ccccc1[C@H](C/C(=N\O)C1CCC(=O)N(C)C1)c1ccc(S(C)(=O)=O)cc1]

### and 5-((R,Z)-1-(hydroxyimino)-3-(4-(methylsulfonyl)phenyl)-3-o-tolylpropyl)-1-methylpiperidin-2-one

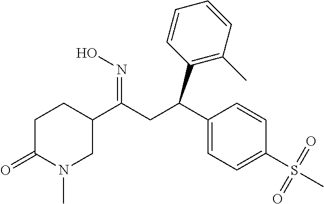 [SMILES: Cc1ccccc1[C@H](C/C(=N/O)C1CCC(=O)N(C)C1)c1ccc(S(C)(=O)=O)cc1]

### Step 1: (E)-Ethyl 3-(4-bromophenyl-2-cyanoacrylate

To a solution of 4-bromobenzaldehyde (106 g, 573 mmol) in toluene (1000 mL) were added ethyl 2-cyanoacetate (71.3 g, 630 mmol) and piperidine (976 mg, 11.5 mmol) and the clear, light brown solution was heated at reflux for 5 h in a 4-neck flask equipped with a Dean-Stark trap, then stirred overnight at room temperature. After cooling the reaction mixture was evaporated, the residue suspended in heptane (500 mL), homogenized in an ultrasound bath for 30 min, stirred for 20 min at 50° C., then the precipitate was collected by filtration. This was dissolved in ethyl acetate (1000 mL) and heptane (500 mL), then slowly concentrated at 50° C. to a volume of approximately 300 mL of solvent. This solution started to crystallize upon standing. The precipitate (96 g) was collected by filtration and washed with heptane/ethyl acetate 9:1 (300 mL). The mother liquor was concentrated at 50° C. until crystallization started. The product was allowed to precipitate over 1 h at room temperature, then collected by filtration to produce a second crop of product (44 g). Total yield: 140 g (87%). Light yellow solid, MS: 299.1 [M+H]⁺.

### Step 2: Ethyl 3-(4-bromophenyl)-2-cyano-3-o-tolylpropanoate

A solution of (E)-ethyl 3-(4-bromophenyl)-2-cyanoacrylate (59.4 g, 212 mmol) in toluene (420 mL) was added over 80 min at 0-5° C. to o-tolylmagnesium chloride solution (1 M in tetrahydrofuran, 276 mL, 276 mmol). The reaction mixture was heated at 85° C. for 1½ h, then poured upon ice water and partitioned between 1 M aq. hydrochloric acid solution and ethyl acetate. The organic layer was washed with brine, dried over magnesium sulfate, filtered, and evaporated to produce the title compound (84.6 g), which was directly used in the next step. Light yellow oil, MS: 370.0 [M−H]⁻.

### Step 3: 3-(4-Bromophenyl)-3-o-tolylpropanoic acid

Sulfuric acid (940 g, 9.59 mol) was added over 30 min under ice cooling to a mixture of ethyl 3-(4-bromophenyl)-2-cyano-3-o-tolylpropanoate (200 g, 494 mmol) in acetic acid (1.06 kg, 17.7 mol), while keeping the internal temperature below 27° C., then the reaction mixture was heated at reflux for 20 h. After cooling to 40° C. ice (500 g) and ethyl acetate (1500 mL) were added, the precipitate collected by filtration and washed with water to produce the title compound (34.5 g). The filtrate was partitioned between ethyl acetate and water, the organic layer was washed with brine, dried over magnesium sulfate, filtered, and evaporated. The residue was stored in the refrigerator for 1 h, then the precipitate was collected by filtration and washed with acetic acid and heptane to afford a second crop of product (96.2 g). Total yield: 130.7 g (83%). Off-white solid, MS: 319.0 [M−H]⁻.

### Step 4: 3-(4-Bromo-phenyl)-N-methoxy-N-methyl-3-o-tolyl-propionamide

To a suspension of 3-(4-bromophenyl)-3-o-tolylpropanoic acid (125.5 g, 393 mmol) in dichloromethane (600 mL) was added 1,1′-carbonyldiimidazole (79.7 g, 491 mmol) portionwise over 5 min. After gas evolution had ceased N,O-dimethylhydroxylamine hydrochloride (42.2 g, 432 mmol) was added in portions over 5 min. The reaction mixture was stirred for 17 h at room temperature, then washed with water. The organic layer was washed with brine, dried over magnesium sulfate, filtered, and evaporated. The crude product was chromatographed (SiO₂; heptane/ethyl acetate 3:1) and the product triturated with heptane to produce the title compound (127 g, 88%). White solid, MS: 362.1 [M+H]⁺.

### Step 5: (R)-3-(4-Bromo-phenyl)-N-methoxy-N-methyl-3-o-tolyl-propionamide and (S)-3-(4-bromo-phenyl)-N-methoxy-N-methyl-3-o-tolyl-propionamide

Separation of 3-(4-bromo-phenyl)-N-methoxy-N-methyl-3-o-tolyl-propionamide (130 g) by chiral HPLC (Reprosil Chiral-NR, heptane/2-propanol 80:20) yielded (R)-3-(4-bromo-phenyl)-N-methoxy-N-methyl-3-o-tolyl-propionamide (60 g, 46%; light yellow oil, MS: 362.1 [M+H]⁺) and (S)-3-(4-bromo-phenyl)-N-methoxy-N-methyl-3-o-tolyl-propionamide (57 g, 44%; light yellow oil, MS: 362.1 [M+H]⁺).

### Step 6: (R)-3-(4-Bromophenyl)-1-(6-methoxypyridin-3-yl)-3-o-tolylpropan-1-one

To a solution of 5-bromo-2-methoxypyridine (16.4 g, 82.8 mmol) in tetrahydrofuran (125 mL) was added dropwise n-butyllithium (1.6 M solution in hexane (51.8 mL, 82.8 mmol) in tetrahydrofuran (38 mL) at a temperature below −70° C. The light brown suspension was stirred for 100 min at −75° C., then a solution of (R)-3-(4-bromophenyl)-N-methoxy-N-methyl-3-o-tolylpropanamide (10.0 g, 27.6 mmol) in tetrahydrofuran (110 mL) was added dropwise to the reaction mixture below −70° C. The reaction mixture was stirred at −75° C. for 1 h, then partitioned between sat. aq. ammonium chloride solution and ethyl acetate. The organic layer was washed with brine, dried over magnesium sulfate, filtered and evaporated. Chromatography (SiO₂, gradient heptane to heptane/ethyl acetate 9:1) afforded the title compound (8.52 g, 75%). Colorless oil, MS: 410.2 [M+H]⁺.

### Step 7: (R)-5-(3-(4-Bromophenyl)-3-o-tolylpropanol)pyridin-2(1H)-one

To a solution of (R)-3-(4-bromophenyl)-1-(6-methoxypyridin-3-yl)-3-o-tolylpropan-1-one (8.52 g, 20.8 mmol) in 1,4-dioxane (280 mL) was added 37% aq. hydrochloric acid solution (38 mL, 0.46 mol) and the resulting mixture heated at 100° C. for 1% h. After cooling the reaction mixture was poured onto ice-water and extracted with ethyl acetate. The organic layer was washed with brine, dried over magnesium sulfate, filtered, and evaporated to afford the title compound (8.8 g), which was used directly in the next step. White solid, MS: 396.1 [M+H]⁺.

### Step 8: (R)-5-(3-(4-Bromophenyl)-3-o-tolylpropanoyl)-1-methylpyridin-2(1H)-one

To a solution of (R)-5-(3-(4-bromophenyl)-3-o-tolylpropanoyl)pyridin-2(1H)-one (8.80 g, 20.7 mmol) in N,N-dimethylacetamide (120 mL) was added iodomethane (3.08 g, 21.7 mmol), followed by potassium carbonate (3.14 g, 22.7 mmol). The reaction mixture was stirred for 16 h at ambient temperature, then another portion of potassium carbonate (571 mg, 4.13 mmol) and iodomethane (586 mg, 4.13 mmol) was added, then after 1 h the reaction mixture was partitioned between water and ethyl acetate, the organic layer washed with brine, dried over magnesium sulfate, filtered, and evaporated. The residue was triturated with tert-butyl methyl ether to afford the title compound (6.63 g, 78%). White solid, MS: 410.2 [M+H]⁺.

### Step 9: 5-[(R-3-(4-Methanesulfonyl-phenyl)-3-o-tolyl-propionyl]-1-methyl-1H-pyridin-2-one

A mixture of L-proline (572 mg, 4.97 mmol) and sodium hydroxide (199 mg, 4.97 mmol) in dimethyl sulfoxide (35 mL) was stirred at room temperature for 30 min, then copper(I) iodide (947 mg, 4.97 mmol) and (R)-5-(3-(4-bromophenyl)-3-o-tolylpropanoyl)-1-methylpyridin-2(1H)-one (2.55 g, 6.21 mmol) and sodium methanesulfinate (5.08 g, 49.7 mmol) were added and the reaction mixture heated at 120° C. for 26 h. After partitioning between water and ethyl acetate, the organic layer was washed with brine, dried over magnesium sulfate, filtered, and evaporated. Chromatography (SiO₂; gradient ethyl acetate/heptane 4:1 to ethyl acetate) afforded the title compound (2.40 g, 94%). Light yellow foam, MS: 410.3 [M+H]⁺.

### Step 10; 1-Methyl-5-((R)-3-(4-(methylsulfonyl)phenyl)-3-o-tolylpropanoyl)piperidin-2-one

As solution of —[(R)-3-(4-methanesulfonyl-phenyl)-3-o-tolyl-propionyl]-1-methyl-1H-pyridin-2-one (156 mg, 0.39 mmol) in methanol (16 mL) was stirred at room temperature under a hydrogen atmosphere (3 bar) in the presence of platinum(IV) oxide (9 mg, 40 μmol), then insoluble material was filtered off and the filtrate evaporated to produce a mixture (ca 1:1) of the title compound and 5-[(R)-1-hydroxy-3-(4-methanesulfonyl-phenyl)-3-o-tolyl-propyl]-1-methyl-piperidin-2-one (151 mg). This mixture was taken up in dichloromethane and treated with 1,1,1-triacetoxy-1,1-dihydro-1,2-benziodoxol-3(1H)-one solution (15 weight-% in dichloromethane 539 mg, 190 μmol) at 0° C. The reaction mixture was allowed to reach room temperature over 1 h, then washed with sat. aq. sodium hydrogencarbonate solution. The organic layer was washed with brine, dried over magnesium sulfate and evaporated. The residue was suspended with dichloromethane and insoluble material was removed by filtration. The filtrate was evaporated and purified by chromatography (SiO₂, gradient dichloromethane to dichloromethane/methanol 9:1) to produce the title compound (132 mg, 84%). White foam, MS: 414.4 [M+H]⁺.

### Step 11: 5-((R)-1-(Hydroxyimino)-3-(4-(methylsulfonyl)phenyl)-3-o-tolylpropyl)-1-methylpiperidin-2-one

To a microwave vial was added 1-methyl-5-((R)-3-(4-(methylsulfonyl)phenyl)-3-o-tolylpropanoyl)piperidin-2-one (188 mg, 0.46 mmol), hydroxylamine hydrochloride (94.8 mg, 1.36 mmol) and sodium bicarbonate (115 mg, 1.36 mmol), ethanol (2 mL) and water (0.2 mL). The vial was capped and heated at 120° C. for 10 min. The reaction mixture was partitioned between ethyl acetate and water. The organic layer was washed with brine, dried over magnesium sulfate and evaporated to afford the title compound (189 mg, 97%) as a mixture of the E and Z stereoisomers (85:15). White foam, MS: 429.3 [M+H]⁺.

### Step 12: 5-((R,E)-1-Hydroxyimino)-3-(4-(methylsulfonyl)phenyl-3-o-tolylpropyl)-1-methylpiperidin-2-one and 5-((R,Z)-1-(hydroxyimino)-3-(4-(methylsulfonyl)phenyl)-3-o-tolylpropyl-1-methylpiperidin-2-one

Separation of 5-((R)-1-(hydroxyimino)-3-(4-(methylsulfonyl)phenyl)-3-O— tolylpropyl)-1-methylpiperidin-2-one (185 mg, 432 μmol) by preparative HPLC (Reprosil Chiral-NR; heptane/ethanol 70:30) afforded 5-((R,E)-1-(hydroxyimino)-3-(4-(methylsulfonyl)phenyl)-3-o-tolylpropyl)-1-methylpiperidin-2-one (example 1; 129 mg, 70%; white foam, MS: 429.4 [M+H]⁺) and 5-((R,Z)-1-(hydroxyimino)-3-(4-(methylsulfonyl)phenyl)-3-o-tolylpropyl)-1-methylpiperidin-2-one (example 2: 24 mg, 13%; light yellow foam, MS: 429.3 [M+H]⁺).

### Examples 3 and 4

### 5-[(R)-1-[(E)-Hydroxyimiao]-3-(4-methanesulfonyl-phenyl)-3-o-tolyl-propyl]-1-methyl-piperidin-2-one, epimer A and epimer B

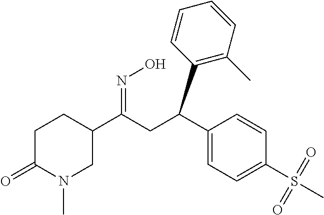 [SMILES: Cc1ccccc1[C@H](C/C(=N\O)C1CCC(=O)N(C)C1)c1ccc(S(C)(=O)=O)cc1]

Separation of 5-((R,E)-1-(hydroxyimino)-3-(4-(methylsulfonyl)phenyl)-3-o-tolylpropyl)-1-methylpiperidin-2-one (example 1; 125 mg, 0.29 mmol) by preparative HPLC (Reprosil Chiral-NR, heptane/ethanol 60:40) afforded 5-[(R)-1-[(E)-hydroxyimino]-3-(4-methanesulfonyl-phenyl)-3-o-tolyl-propyl]-1-methyl-piperidin-2-one, epimer A (48 mg, 38%; brown foam, t_{R}=83 min; MS: 429.3 [M+H]⁺) and 5-[(R)-1-[(E)-hydroxyimino]-3-(4-methanesulfonyl-phenyl)-3-o-tolyl-propyl]-1-methyl-piperidin-2-one, epimer B (43 mg, 34%; t_{R}=⁸⁹ min; light brown foam, MS: 429.3 [M+H]⁺).

### Example 5

### Sodium 1-(4-((1R,E)-3-(hydroxyimino)-3-(1-methyl-6-oxopiperidin-3-yl)-1-o-tolylpropyl)phenyl)piperidine-4-carboxylate

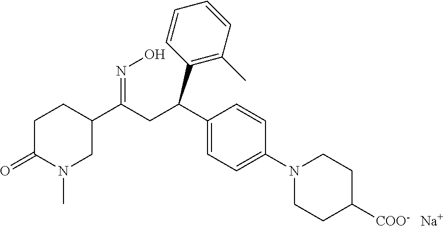

### Step 1: (R)-Ethyl 1-(4-(3-(1-methyl-6-oxo-1,6-dihydropyridin-3-yl)-3-oxo-1-o-tolylpropyl)phenylpiperidine-4-carboxylate

To a solution of (R)-5-(3-(4-bromophenyl)-3-o-tolylpropanoyl)-1-methylpyridin-2(1H)-one (examples 1 and 2, step 8; 500 mg, 1.22 mmol) in toluene (7 mL) were added ethyl piperidine-4-carboxylate (293 mg, 1.83 mmol), sodium tert-butoxide (234 mg, 2.44 mmol), tris(dibenzylideneacetone)dipalladium(0) (22.3 mg, 24.4 μmol) and 2-dicyclohexylphosphino-2′,4′,6′-triisopropylbiphenyl (23.7 mg, 48.7 μmol), and the reaction mixture was stirred for 1 h at 85° C., then was evaporated. Chromatography (SiO₂, gradient dichloromethane to dichloromethane/methanol 19:1) produced the title compound (174 mg; light yellow foam, MS: 487.4 [M+H]⁺) and (R)-1-methyl-5-(3-phenyl-3-o-tolylpropanoyl)pyridin-2(1H)-one (88 mg; off-white solid, MS: 332.2 [M+H]⁺).

### Step 2: Ethyl 1-(4-((1R)-3-(1-methyl-6-oxopiperidin-3-yl-3-oxo-1-o-tolylpropyl)-phenyl)piperidine-4-carboxylate

The title compound was produced in analogy to examples 1 and 2, step 10 from (R)-ethyl 1-(4-(3-(1-methyl-6-oxo-1,6-dihydropyridin-3-yl)-3-oxo-1-o-tolylpropyl)phenyl)-piperidine-4-carboxylate. Yellow gum, MS: 491.4 [M+H]⁺.

### Step 3: Sodium 1-(4-((1R)-3-(1-methyl-6-oxopiperidin-3-yl)-3-oxo-1-o-tolylpropyl)-phenyl)piperidine-4-carboxylate

To a solution of ethyl 1-(4-((1R)-3-(1-methyl-6-oxopiperidin-3-yl)-3-oxo-1-o-tolylpropyl)phenyl)piperidine-4-carboxylate (35 mg, 71 μmol) in tetrahydrofuran (1 mL) and methanol (0.4 mL) was added 1 M aq. sodium hydroxide solution (0.7 mL, 0.7 mmol) at room temperature, then after 1 h the reaction mixture was evaporated. Chromatography (SiO₂; gradient dichloromethane to dichloromethane/methanol 4:1) afforded the title compound (31 mg, 90%). Light brown foam MS: 463.3 [M+H]⁺.

### Step 4: Sodium 1-(4-((1R,E)-3-(hydroxyimino-3-(1-methyl-6-oxpiperidin-3-yl)-1-o-tolylpropyl)phenyl)piperidine-4-carboxylate

To a microwave vial was added sodium 1-(4-((1R)-3-(1-methyl-6-oxopiperidin-3-yl)-3-oxo-1-o-tolylpropyl)phenyl)piperidine-4-carboxylate (27 mg, 56 μmol), hydroxylamine hydrochloride (11.6 mg, 167 μmol) and sodium hydrogencarbonate (23.4 mg, 279 μmol) in ethanol (0.5 mL) and water (50 μl). The vial was capped and heated at 120° C. for 20 min, then the reaction mixture was evaporated. Chromatography (SiO₂, gradient dichloromethane to dichloromethane/methanol 9:1) afforded the title compound (22 mg, 79%). Light red solid MS: 478.3 [M+H]⁺.

### Example 6

### 5-((R,E)-1-(Hydroxyimino)-3-phenyl-3-o-tolylpropyl)-1-methylpiperidin-2-one

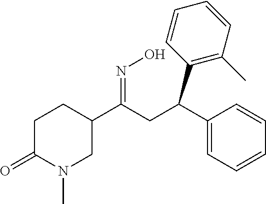 [SMILES: Cc1ccccc1[C@H](C/C(=N\O)C1CCC(=O)N(C)C1)c1ccccc1]

### Step 1: 1-Methyl-5-((R)-3-phenyl-3-o-tolylpropanoyl)piperidin-2-one

The title compound was produced in analogy to examples 1 and 2, step 10 from (R)-1-methyl-5-(3-phenyl-3-o-tolylpropanoyl)pyridin-2(1H)-one (example 5, step 1). Colorless gum, MS: 336.4 [M+H]⁺.

### Step 2: 5-((R,E)-1-(Hydroxyimino)-3-phenyl-3-o-tolylpropyl-1-methylpiperidin-2-one

The title compound was produced in analogy to examples 1 and 2, step 11 from 1-methyl-5-((R)-3-phenyl-3-o-tolylpropanoyl)piperidin-2-one. White foam, MS: 351.3 [M+H]⁺.

### Example 7

### 4′-((1R,E)-3-(Hydroxyimino)-3-(1-methyl-6-oxopiperidin-3-yl)-1-o-tolylpropyl)phenyl-4-carboxylic acid

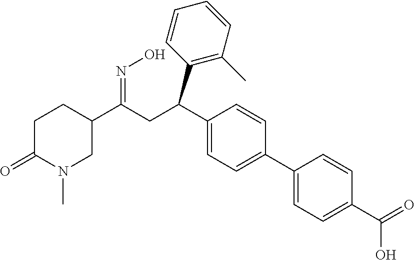 [SMILES: Cc1ccccc1[C@H](C/C(=N\O)C1CCC(=O)N(C)C1)c1ccc(-c2ccc(C(=O)O)cc2)cc1]

### Step 1: (R)-4′-(3-(1-methyl-6-oxo-1,6-dihydropyridin-3-yl)-3-oxo-1-o-tolylpropyl)biphenyl-4-carboxylic acid

To a solution of (R)-5-(3-(4-bromophenyl)-3-o-tolylpropanoyl)-1-methylpyridin-2(1H)-one (examples 1 and 2, step 8; 0.3 g, 731 μmol) in 1,4-dioxane (2.4 mL) were added 4-carboxyphenylboronic acid (182 mg, 1.1 mmol), [1,1′-bis(diphenylphosphino)ferrocene]-dichloropalladium(II) dichloromethane adduct (30 mg, 37 μmol), water (1.8 mL) and 2 M aq. sodium carbonate solution (1.1 mL, 2.2 mmol) and the resulting dark brown mixture was heated for 2 h at 80° C. The reaction mixture was partitioned between 10% aq. citric acid solution and ethyl acetate. The organic layer was washed with brine, dried over magnesium sulfate, filtered, and evaporated. Chromatography (SiO₂; heptane/dichloromethane/methanol (100:0:0 to 0:85:15) produced the title compound (291 mg, 88%). Light brown solid, MS: 452.2 [M+H]⁺.

### Step 2: 4′-((R)-3-(1-methyl-6-oxopiperidin-3-yl)-3-oxo-1-o-tolylpropyl)biphenyl-4-carboxylic acid

The title compound was produced in analogy to examples 1 and 2, step 10 from (R)-4′-(3-(1-methyl-6-oxo-1,6-dihydropyridin-3-yl)-3-oxo-1-o-tolylpropyl)biphenyl-4-carboxylic acid. White foam, MS: 454.3 [M−H]⁻.

### Step 3: 4′-((1R,E)-3-(hydroxyimino)-3-(1-methyl-6-oxopiperidin-3-yl)-o-tolylpropyl)-biphenyl-4-carboxylic acid

The title compound was produced in analogy to examples 1 and 2, step 11 from 4′-((1R)-3-(1-methyl-6-oxopiperidin-3-yl)-3-oxo-1-o-tolylpropyl)biphenyl-4-carboxylic acid. White solid, MS: 469.4 [M−H]⁻.

### Example 8

### (E)-1-(4-(3-(4-Bromophenyl-(hydroxyimino)-3-o-tolylpropyl)piperidin-1-yl)ethanone

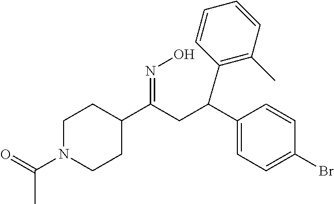 [SMILES: CC(=O)N1CCC(/C(CC(c2ccc(Br)cc2)c2ccccc2C)=N/O)CC1]

### Step 1: tert-Butyl 4-(2-(dimethoxyphosphoryl)acetyl)piperidine-1 carboxylate

To a solution of 1-tert-butyl 4-methyl piperidine-1,4-dicarboxylate (1.65 g, 6.58 mmol) and dimethyl methylphosphonate (926 mg, 7.24 mmol) in tetrahydrofuran (10 mL) was added lithium diisopropylamide (2 M in tetrahydroduran, 6.9 mL, 13.8 mmol) at between −5° C. and 0° C., then after 10 min the pH was adjusted to ca. 6 by careful addition of 4 M aq. hydrochloric acid solution. The reaction mixture was then extracted with dichloromethane, the organic layer washed with brine, dried over magnesium sulfate and evaporated. Chromatography (SiO₂, gradient ethyl acetate to ethyl acetate/methanol 9:1) afforded the title compound (2.07 g, 94%). Yellow oil MS: 336.2 [M+H]⁺.

### Step 2: (E)-tert-Butyl 4-(3-(4-bromophenyl)acryloyl)piperidine-1-carboxylate

To a solution of tert-butyl 4-(2-(dimethoxyphosphoryl)acetyl)piperidine-1-carboxylate (1.00 g, 2.98 mmol) in ethanol (10 mL) was added potassium carbonate (824 mg, 5.96 mmol) and 4-bromobenzaldehyde (557 mg, 2.98 mmol). The reaction mixture was heated at 90° C. for 30 min, then partitioned between ethyl acetate and water. The organic layer was washed with brine, dried over magnesium sulfate, filtered, and evaporated. The residue was triturated in heptane/ethyl acetate 9:1 to afford the title compound (941 mg, 80%). White solid, MS: 394.1 [M+H]⁺.

### Step 3: tert-Butyl 4-(3-(4-bromophenyl)-3-o-tolylpropanoyl)piperidine-1-carboxylate

To a suspension of copper(I) iodide (23 mg, 0.12 mmol) in tetrahydrofuran (5 mL) was added at 0° C. o-tolylmagnesium bromide solution (2 M in diethyl ether, 1.33 mL, 2.66 mmol) to give a light brown suspension. The reaction mixture was stirred at this temperature for 1 h, then (E)-tert-butyl 4-(3-(4-bromophenyl)acryloyl)piperidine-1-carboxylate (477 mg, 1.21 mmol) was added, then after 75 min the reaction mixture was partitioned between sat. aq. ammonium chloride solution and ethyl acetate. The organic layer was washed with brine, dried over magnesium sulfate, filtered, and evaporated. The residue was dissolved in heptane and a few drops of ethyl acetate. After a few minutes a white solid precipitated, which was collected by filtration to afford the title compound (442 mg, 75%). Chromatography of the mother liquor (SiO₂, gradient heptane to heptane/ethyl acetate 1:1) afforded a second crop of product (73 mg, 12%). White solid, MS: 486.2 [M+H]⁺.

### Step 4: 3-(4-Bromophenyl)-1-(piperidine-4-yl)-3-o-tolylpropan-1-one

To a colorless solution of tert-butyl 4-(3-(4-bromophenyl)-3-o-tolylpropanoyl)-piperidine-1-carboxylate (386 mg, 794 μmol) in 1,4-dioxane (3.5 mL) was added hydrogen chloride solution (4 M in 1,4-dioxane, 4.0 mL, 16 mmol), then after 2 h the reaction mixture was concentrated in vacuo. The residue was partitioned between ethyl acetate and sat. aq. sodium hydrogencarbonate solution. The organic layer was washed with brine, dried over magnesium sulfate, filtered, and evaporated. Chromatography (SiO₂; gradient dichloromethane to dichloromethane/methanol/25% aq. ammonia solution 90:10:0.25) afforded the title compound (284 mg, 93%). White foam, MS: 386.1 [M+H]⁺.

### Step 5: 1-(1-Acetylpiperidin-4-yl)-3-(4-bromophenyl-3-o-tolylpropan-1-one

To a solution of 3-(4-bromophenyl)-1-(piperidin-4-yl)-3-o-tolylpropan-1-one (82 mg, 0.21 mmol) in acetonitrile (1 mL) was added N,N-diisopropylethylamine (68.6 mg, 0.53 mmol) and acetyl chloride (25 mg, 0.32 mmol), then after 1 h the reaction mixture was partitioned between sat. aq. ammonium chloride solution and ethyl acetate. The organic layer was washed with brine, dried over magnesium sulfate, filtered and evaporated to afford the title compound (86 mg; 95%). White foam, MS:428.2 [M+H]⁺.

### Step 6: (E)-1-(4-(3-(4-bromophenyl)-1-(hydroxyimino-3-o-tolylpropyl)piperidin-1-yl)ethanone

The title compound was produced in analogy to examples 1 and 2, step 11 from 1-(1-acetylpiperidin-4-yl)-3-(4-bromophenyl)-3-o-tolylpropan-1-one. White foam, MS: 443.1 [M+H]⁺.

### Example 9

### (E)-3-(4-Bromophenyl)-1-(tetrahydro-2H-pyran-4-yl)-3-o-tolylpropan-1-one oxime

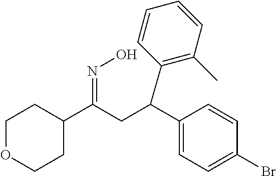 [SMILES: Cc1ccccc1C(C/C(=N\O)C1CCOCC1)c1ccc(Br)cc1]

### Step 1: Dimethyl 2-oxo-2-(tetrahydro-2H-pyran-4-yl)ethylphosphonate

The title compound was produced in analogy to example 8, step 1 from methyl tetrahydro-2H-pyran-4-carboxylate and dimethyl methylphosphonate. Light yellow liquid, MS: 237.1 [M+H]⁺.

### Step 2: (E)-3-(4-bromophenyl)-1-(tetrahydro-2H-pyran-4-yl)prop-2-en-1-one

The title compound was produced in analogy to example 8, step 2 from dimethyl 2-oxo-2-(tetrahydro-2H-pyran-4-yl)ethylphosphonate and 4-bromobenzaldehyde. White solid, MS: 295.1 [M+H]⁺.

### Step 3: 3-(4-Bromophenyl)-1-(tetrahydro-2H-pyran-4-yl)-3-o-tolylpropan-1-one

The title compound was produced in analogy to example 8, step 3 from (E)-3-(4-bromophenyl)-1-(tetrahydro-2H-pyran-4-yl)prop-2-en-1-one and o-tolylmagnesium chloride. Light yellow oil, MS: 387.2 [M+H]⁺.

### Step 4: (E)-3-(4-Bromophenyl)-1-(tetrahydro-2H-pyran-4-yl)-3-o-tolylpropan-1-one oxime

The title compound was produced in analogy to examples 1 and 2, step 11 from 3-(4-bromophenyl)-1-(tetrahydro-2H-pyran-4-yl)-3-o-tolylpropan-1-one. White foam, MS: 402.3 [M+H]⁺.

### Example 10

### (E)-3-(4-(Methylsulfonyl)phenyl)-3-phenyl-1-(tetrahydro-2H-pyran-4-yl)propan-1-one oxime

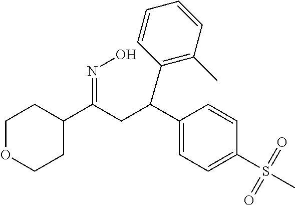 [SMILES: Cc1ccccc1C(C/C(=N\O)C1CCOCC1)c1ccc(S(C)(=O)=O)cc1]

### Step 1: (E)-3-(4-(methylsulfonyl)phenyl-1-(tetrahydro-2H-pyran-4-yl)prop-2-en-1-one

The title compound was produced in analogy to example 8, step 2 from dimethyl 2-oxo-2-(tetrahydro-2H-pyran-4-yl)ethylphosphonate (example 9, step 1) and 4-(methylsulfonyl)-benzaldehyde. White solid, MS: 295.2 [M+H]⁺.

### Step 2: 3-(4-(methylsulfonyl)phenyl)-3-phenyl-1-(tetrahydro-2H-pyran-4-yl)propan-1-one

A mixture of (E)-3-(4-(methylsulfonyl)phenyl)-1-(tetrahydro-2H-pyran-4-yl)prop-2-en-1-one (132 mg, 448 μmol), phenylboronic acid (109 mg, 897 μmol), palladium(II) acetate (5.03 mg, 22.4 μmol), triphenylphosphine (11.8 mg, 44.8 μmol) and cesium carbonate (146 mg, 448 μmol) in toluene (2 mL) and chloroform (0.01 mL) was heated at 80° C. overnight, then partitioned between dichloromethane and water. The organic layer was washed with brine, dried over magnesium sulfate, filtered, and evaporated. Chromatography (SiO₂; gradient dichloromethane to dichloromethane/methanol 9:1) produced the title compound (23 mg, 14%). White foam, MS:373.2 [M+H]⁺.

### Step 3: (E)-3-(4-(Methylsulfonyl)phenyl)-3-phenyl-1-(tetrahydro-2H-pyran-4-yl)propan-1-one oxime

The title compound was produced in analogy to examples 1 and 2, step 11 from 3-(4-(methylsulfonyl)phenyl)-3-phenyl-1-(tetrahydro-2H-pyran-4-yl)propan-1-one. White foam, MS: 388.1 [M+H]⁺.

### Example 11

### (E)-3-(4-Bromophenyl)-1-(tetrahydro-2H-thiopyran-4-yl)-3-o-tolylpropan-1-one oxime

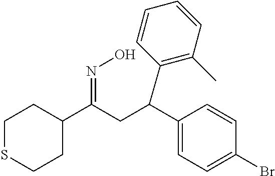 [SMILES: Cc1ccccc1C(C/C(=N\O)C1CCSCC1)c1ccc(Br)cc1]

### Step 1: Dimethyl 2-oxo-2-(tetrahydro-2H-thiopyran-4-yl)ethylphosphonate

The title compound was produced in analogy to example 8, step 1 from methyl tetrahydro-2H-thiopyran-4-carboxylate and dimethyl methylphosphonate. Light yellow oil, MS: 253.1 [M+H]⁺.

### Step 2: (E)-3-(4-Bromophenyl)-1-(tetrahydro-2H-thiopyran-4-yl)prop-2-en-1-one

The title compound was produced in analogy to example 8, step 2 from dimethyl 2-oxo-2-(tetrahydro-2H-thiopyran-4-yl)ethylphosphonate and 4-bromobenzaldehyde. White solid, MS: 312.9 [M+H]⁺.

### Step 3: 3-(4-Bromophenyl)-1-(tetrahydro-2H-thiopyran-4-yl)-3-o-tolylpropan-1-one

The title compound was produced in analogy to example 8, step 3 from (E)-3-(4-bromophenyl)-1-(tetrahydro-2H-thiopyran-4-yl)prop-2-en-1-one and o-tolylphenylmagnesium bromide. Colorless oil, MS: 403.2 [M+H]+.

### Step 4: (E)-3-(4-Bromophenyl-1-(tetrahydro-2H-thiopyran-4-yl)-3-o-tolylpropan-1-one oxime

The title compound was produced in analogy to examples 1 and 2, step 11 from 3-(4-bromophenyl)-1-(tetrahydro-2H-thiopyran-4-yl)-3-o-tolylpropan-1-one. White solid, MS: 418.2 [M+H]⁺.

### Example 12

### 3-(4-Bromo-phenyl)-1-(1,1-dioxo-hexahydro-thiopyran-4-yl)-3-o-tolyl-propan-1-one oxime

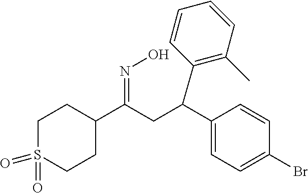 [SMILES: Cc1ccccc1C(C/C(=N\O)C1CCS(=O)(=O)CC1)c1ccc(Br)cc1]

### Step 1: 3-(4-Bromo-phenyl)-1-(1,1-dioxo-hexahydro-thiopyran-4-yl)-3-o-tolyl-propan-1-one

To a solution of 3-(4-bromophenyl)-1-(tetrahydro-2H-thiopyran-4-yl)-3-o-tolylpropan-1-one (example 11, step 3; 300 mg, 707 μmol) in formic acid (3 mL) was added 30% aq. hydrogen peroxide solution (0.31 mL, 3.5 mmol) at ambient temperature, then after 2 h the reaction mixture was partitioned between 1 M aq. sodium carbonate solution and ethyl acetate. The organic layer was washed with brine, dried over magnesium sulfate, filtered, and evaporated in vacuo. The residue was purified by chromatography (SiO₂, gradient heptane to heptane/ethyl acetate 1:3) to afford the title compound (277 mg, 90%). White solid, MS: 435.2 [M+H]⁺.

### Step 2: 3-(4-Bromophenyl)-1-(1,1-dioxo-hexahydro-thiopyran-4-yl)-3-o-tolyl-propan-1-one oxime

The title compound was produced in analogy to examples 1 and 2, step 11 from 3-(4-bromo-phenyl)-1-(1,1-dioxo-hexahydro-thiopyran-4-yl)-3-o-tolyl-propan-1-one. White solid, MS: 450.1 [M+H]⁺.

### Example 13

### (E)-3-(4-Bromophenyl)-1-(trans-4-hydroxycyclohexyl)-3-o-tolylpropan-1-one oxime

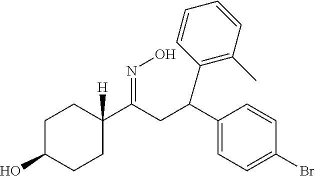 [SMILES: Cc1ccccc1C(C/C(=N\O)[C@H]1CC[C@H](O)CC1)c1ccc(Br)cc1]

### Step 1: Ethyl 4-(tetrahydro-2H-pyran-2-yloxy)cyclohexanecarboxylate

To a solution of ethyl 4-hydroxycyclohexanecarboxylate (5.00 g, 29.0 mmol) in dichloromethane (30 mL) was added 3,4-dihydro-2H-pyran (2.52 g, 29.0 mmol) and Amberlyst® 15 (0.5 g). The reaction mixture was stirred at room temperature for 72 h, then another portion 3,4-dihydro-2H-pyran (1.89 g, 21.8 mmol) and Amberlyst® 15 (0.5 g) was added, then after 5 h the reaction mixture was washed with sat aq. sodium hydrogencarbonate solution. The organic layer was washed with brine, dried over magnesium sulfate, filtered, and evaporated to afford the title compound as a yellow oil (9 g), which was used directly in the next step.

### Step 2: Dimethyl 2-oxo-2-(4-(tetrahydro-2H-pyran-2-yloxy)cyclohexyl)ethylphosphonate

The title compound was produced in analogy to example 8, step 1 from ethyl 4-(tetrahydro-2H-pyran-2-yloxy)cyclohexanecarboxylate and dimethyl methylphosphonate. Yellow oil, MS: 334 [M]⁺.

### Step 3: (E)-3-(4-Bromophenyl)-1-(trans-4-(tetrahydro-2H-pyran-2-yloxy)cyclohexyl)prop-2-en-1-one

To a solution of dimethyl 2-oxo-2-(4-(tetrahydro-2H-pyran-2-yloxy)cyclohexyl)ethyl-phosphonate (1.05 g, 3.14 mmol) in ethanol (10 mL) was added potassium carbonate (868 mg, 6.28 mmol) and 4-bromobenzaldehyde (581 mg, 3.14 mmol). The reaction mixture was heated at 90° C., then after 50 min partitioned between water and ethyl acetate. The organic layer was washed with brine, dried over magnesium sulfate, filtered, and evaporated. The residue was triturated in heptane to produce the title compound (413 mg, 33%; white solid, MS: 394.0 [M+H]⁺). The mother liquor was chromatographed (SiO₂; heptane-ethyl acetate gradient) to produce (E)-3-(4-bromophenyl)-1-(4-(tetrahydro-2H-pyran-2-yloxy)cyclohexyl)prop-2-en-1-one (587 mg, 47%; white solid, MS: 394.0 [M+H]⁺) as a 1:1 mixture of the cis and trans stereoisomers.

### Step 4: 3-(4-bromophenyl)-1-(trans-4-(tetrahydro-2H-pyran-2-yloxy)cyclohexyl)-3-o-tolylpropan-1-one

The title compound was produced in analogy to example 8, step 3 from (E)-3-(4-bromophenyl)-1-(trans-4-(tetrahydro-2H-pyran-2-yloxy)cyclohexyl)prop-2-en-1-one and o-tolylmagnesium bromide. Light yellow oil, MS: 502.2 [M+NH₄]⁺.

### Step 5: 3-(4-bromophenyl)-1-(trans-4-hydroxycyclohexyl)-3-o-tolylpropan-1-one

To a solution of 3-(4-bromophenyl)-1-(trans-4-(tetrahydro-2H-pyran-2-yloxy)cyclohexyl)-3-o-tolylpropan-1-one (462 mg, 0.95 mmol) in methanol (5 mL) was added trifluoroacetic acid (109 mg, 0.95 mmol) at room temperature, then after 90 min the reaction mixture was partitioned between sat. aq. sodium hydrogencarbonate solution and ethyl acetate. The organic layer was washed with brine, dried over magnesium sulfate, filtered, and evaporated. Chromatography (SiO₂; gradient dichloromethane to dichloromethane/methanol/25% ammonia solution 95:5:0.25) afforded the title compound (346 mg, 91%). White foam, MS: 418.2 [M+NH₄]⁺.

### Step 6: (E)-3-(4-Bromophenyl)-1-(trans-4-hydroxycyclohexyl)-3-o-tolylpropan-1-one oxime

To a microwave vial was added 3-(4-bromophenyl)-1-(trans-4-hydroxycyclohexyl)-3-o-tolylpropan-1-one (338 mg, 842 μmol), hydroxylamine hydrochloride (176 mg, 2.53 mmol) and sodium hydrogencarbonate (212 mg, 2.53 mmol), ethanol (4 mL), and water (0.2 mL). The vial was capped and heated at 120° C. for 15 min. The reaction mixture was partitioned between ethyl acetate and water. The organic layer was washed with brine, dried over magnesium sulfate, filtered, and evaporated. Chromatography (SiO₂; gradient dichloromethane to dichloromethane/methanol/25% aq. ammonia solution 95:5:0.25) afforded the title compound (255 mg, 73%). and (Z)-3-(4-bromophenyl)-1-(trans-4-hydroxycyclohexyl)-3-o-tolylpropan-1-one oxime (45 mg, 13%; White foam, MS: 416.2 [M+H]⁺).

### Example 14

### (E)-4-(3-(4-bromophenyl)-1-(hydroxyimino)-3-o-tolylpropyl)cyclohexanone

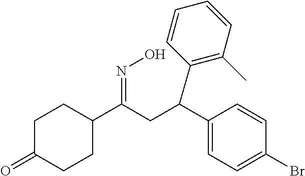 [SMILES: Cc1ccccc1C(C/C(=N\O)C1CCC(=O)CC1)c1ccc(Br)cc1]

To a solution of (E)-3-(4-bromophenyl)-1-(trans-4-hydroxycyclohexyl)-3-o-tolylpropan-1-one oxime (example 13; 128 mg, 0.31 mmol) in toluene (10 mL) was added 1-methyl-4-piperidone (3.34 g, 28.9 mmol). The reaction mixture was heated at reflux until 1-2 mL toluene had condensed in a Dean-Stark trap, then aluminum isopropoxide (113 mg, 0.55 mmol) was added. The reaction mixture was stirred at reflux for 2 h, then another portion of aluminum isopropoxide (63 mg, 0.31 mmol) was added, then after 16 h the reaction mixture was partitioned between sat. aq. ammonium chloride solution and ethyl acetate. The organic layer was washed with water and brine, dried over magnesium sulfate, filtered, and evaporated. Chromatography (SiO₂, ethyl acetate/heptane 1:1) afforded the title compound (31 mg, 24%). Yellow foam; MS: 414.2 [M+H]⁺.

### Examples 15 and 16

### (E)-1-(4-Hydroxycyclohexyl)-3-(4-(methylsulfonyl)phenyl)-3-o-tolylpropan-1-one oxime

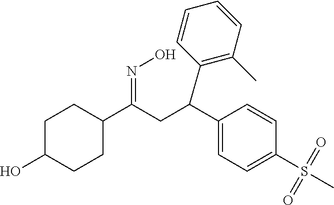 [SMILES: Cc1ccccc1C(C/C(=N\O)C1CCC(O)CC1)c1ccc(S(C)(=O)=O)cc1]

### and (Z)-1-(4-hydroxycyclohexyl)-3-(4-(methylsulfonyl)phenyl)-3-o-tolylpropan-1-one oxime

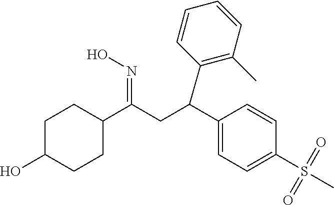 [SMILES: Cc1ccccc1C(C/C(=N/O)C1CCC(O)CC1)c1ccc(S(C)(=O)=O)cc1]

### Step 1: 3-(4-(methylsulfonylphenyl)-1-(4-(tetrahydro-2H-pyran-2-yloxy)cyclohexyl)-3-o-tolylpropan-1-one

The title compound was produced in analogy to example 1, step 9 from 3-(4-bromo-phenyl)-1-(4-(tetrahydro-2H-pyran-2-yloxy)cyclohexyl)-3-o-tolylpropan-1-one (example 13, step 4) and sodium methanesulfinate. White foam, MS: 485.4 [M+H]⁺.

### Step 2: 1-(4-Hydroxycyclohexyl-3-(4-(methylsulfonyl)phenyl)-3-o-tolylpropan-1-one

The title compound was produced in analogy to example 13 step 5 from 3-(4-(methyl-sulfonyl)phenyl)-1-(4-(tetrahydro-2H-pyran-2-yloxy)cyclohexyl)-3-o-tolylpropan-1-one. Light yellow gum, MS: 401.3 [M+H]⁺.

### Stet 3: (E)-1-(4-Hydroxycyclohexyl)-3-(4-(methylsulfonyl)phenyl)-3-o-tolylpropan-1-one oxime and (Z)-1-(4-hydroxycyclohexyl)-3-(4-(methylsulfonyl)phenyl)-3-o-tolylpropan-1-one oxime

To a microwave vial was added 1-(4-hydroxycyclohexyl)-3-(4-(methylsulfonyl)phenyl)-3-o-tolylpropan-1-one (169 mg, 422 μmol, Eq: 1.00), sodium hydrogencarbonate (106 mg, 1.27 mmol) and hydroxylamine hydrochloride (88.0 mg, 1.27 mmol), ethanol (2 mL) and water (0.2 mL). The vial was capped and heated at 120° C. for 15 min, then the reaction mixture was partitioned between water and ethyl acetate. The organic layer was washed with brine, dried over magnesium sulfate, filtered, and evaporated. Chromatography (SiO₂; gradient dichloromethane to dichloromethane/methanol/25% aq. ammonia solution 95:5:0.25) produced (E)-1-(4-hydroxycyclohexyl)-3-(4-(methylsulfonyl)phenyl)-3-o-tolylpropan-1-one oxime (135 mg, 77%; white foam, MS: 416.4 [M+H]⁺) and (Z)-1-(4-hydroxycyclohexyl)-3-(4-(methylsulfonyl)-phenyl)-3-o-tolylpropan-1-one oxime (39 mg, 22%; light yellow foam, MS: 416.4 [M+H]⁺).

### Example 17

### (E)-1-((1r,4r)-4-Hydroxy-4-methylcyclohexyl)-3-(4-(methylsulfonyl)phenyl)-3-o-tolylpropan-1-one oxime

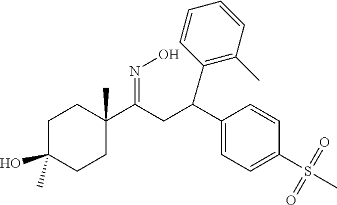 [SMILES: Cc1ccccc1C(C/C(=N\O)[C@]1(C)CC[C@@](C)(O)CC1)c1ccc(S(C)(=O)=O)cc1]

### Step 1: 44-(2-(4-(4-(Methylsulfonyl)phenyl)-2-o-tolylethyl)-1,3-dioxolan-2-yl)cyclohexanol

To a solution of 1-(4-hydroxycyclohexyl)-3-(4-(methylsulfonyl)phenyl)-3-o-tolylpropan-1-one (examples 15 and 16, step 2; 1.26 g, 3.14 mmol) and 1,2-bis(trimethylsilyloxy)ethane (810 mg, 3.92 mmol) in dichloromethane (15 mL) was added dropwise a solution of trimethylsilyl trifluoromethanesulfonate (140 mg, 628 μmol) in dichloromethane (2 mL) at 0° C. The yellow solution was allowed to warm to room temperature over 6 h, then kept in the refrigerator for 36 h. After addition of triethylamine (54 mg, 0.64 mmol) the reaction mixture was partitioned between sat. aq. sodium hydrogencarbonate solution and dichloromethane. The organic layer was dried over magnesium sulfate, filtered, and evaporated. Chromatography (SiO₂; gradient dichloromethane to dichloromethane/methanol/25% aq. ammonia solution 95:5:0.25) produced the title compound (655 mg, 44%; light yellow foam, MS: 445.2 [M+H]⁺) and trimethyl(4-(2-(2-(4-(methylsulfonyl)phenyl)-2-o-tolylethyl)-1,3-dioxolan-2-yl)cyclohexyloxy)silane (715 mg, 39%; yellow foam, MS: 517.3 [M+H]J), which could be converted to the title compound by treatment with potassium carbonate in methanol at room temperature.

### Step 2: 4-(2-(4-(Methylsulfonyl)phenyl)-2-o-tolylethyl)-1,3-dioxolan-2-yl)cyclohexanone

To a solution of 4-(2-(2-(4-(methylsulfonyl)phenyl)-2-o-tolylethyl)-1,3-dioxolan-2-yl)cyclohexanol (197 mg, 332 μmol) in dichloromethane (2 mL) was added 1,1,1-triacetoxy-1,1-dihydro-1,2-benziodoxol-3(1H)-one solution (15% in dichloromethane, 1.22 mL, 432 μmol), then after 5 h the reaction mixture was partitioned between dichloromethane and sat. aq. sodium hydrogencarbonate solution. The organic layer was washed with brine, dried over magnesium sulfate, filtered, and evaporated. Chromatography (SiO₂; gradient dichloromethane to dichloromethane/methanol/25% aq. ammonia solution 95:5:0.25 afforded the title compound (181 mg, 95%). White foam, MS: 443.3 [M+H]⁺.

### Step 3: (1r,4r)-1-Methyl-4-(2-(2-(4-(methylsulfonyl)phenyl)-2-o-tolylethyl)-1,3-dioxolan-2-yl)cyclohexanol

To a solution of 4-(2-(2-(4-(methylsulfonyl)phenyl)-2-o-tolylethyl)-1,3-dioxolan-2-yl)cyclohexanone (171 mg, 298 μmol) in tetrahydrofuran (2 mL) was added methylmagnesium bromide solution (3 M in tetrahydrofuran, 139 μl, 446 μmol) in tetrahydrofuran (0.5 mL) dropwise at 0° C., then after 45 min the reaction mixture was partitioned between sat. aq. ammonium chloride solution and ethyl acetate. The organic layer was washed with brine, dried over magnesium sulfate, filtered, and evaporated. Chromatography (SiO₂; heptane-ethyl acetate gradient) the title compound (43 mg, 32%; colorless gum, MS: 476.2 [M+NH₄]⁺) and (1s,4s)-1-methyl-4-(2-(2-(4-(methylsulfonyl)-phenyl)-2-o-tolylethyl)-1,3-dioxolan-2-yl)cyclohexanol (69 mg, 50%; white foam, MS: 476.2 [M+NH₄]⁺).

### Step 4: 1-((1r,4r)-4-hydroxy-4-methylcyclohexyl)-3-(4-(methylsulfonyl)phenyl)-3-o-tolylpropan-1-one

To a solution of (1r,4r)-1-methyl-4-(2-(2-(4-(methylsulfonyl)phenyl)-2-o-tolylethyl)-1,3-dioxolan-2-yl)cyclohexanol (43 mg, 93.8 μmol, Eq: 1.00) in acetone (1 mL) was added at room temperature bis(acetonitrile)dichloropalladium(II) (1 mg, 5 μmol), then after 20 h another portion of bis(acetonitrile)dichloropalladium(II) (5 mg, 20 μmol), then after 6 h the reaction mixture was partitioned between brine and ethyl acetate. The organic layer was dried over magnesium sulfate, filtered, and evaporated to produce the title compound (39 mg, 100%). Light yellow foam, MS: 432.3 [M+NH₄]⁺.

### Step 5: (E)-1-((1r,4r)-4-Hydroxy-4-methylcyclohexyl)-3-(4-(methylsulfonyl)phenyl)-3-o-tolylpropan-1-one oxime

The title compound was produced in analogy to examples 1 and 2, step 11 from 1-((1r,4r)-4-hydroxy-4-methylcyclohexyl)-3-(4-(methylsulfonyl)phenyl)-3-o-tolylpropan-1-one. White foam, MS: 430.4 [M+H]⁺.

### Example 18

### (E)-3-(4-Bromophenyl)-1-(3-hydroxycyclobutyl)-3-o-tolylpropan-1-one oxime

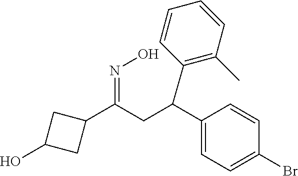 [SMILES: Cc1ccccc1C(C/C(=N\O)C1CC(O)C1)c1ccc(Br)cc1]

### Step 1: Ethyl 3-(tetrahydro-2H-pyran-2-yloxy)cyclobutanecarboxylate

The title compound was produced in analogy to example 13, step 1 from ethyl 3-hydroxycyclobutanecarboxylate and 3,4-dihydro-2H-pyran.

### Step 2: 2-Oxo-2-(3-(tetrahydro-2H-pyran-2-yloxy)cyclobutyl)ethylphosphonate

The title compound was produced in analogy to example 8 step 1 from ethyl 3-(tetrahydro-2H-pyran-2-yloxy)cyclobutanecarboxylate and dimethyl methyl phosphonate. Orange liquid, MS: 324.2 [M+NH₄]⁺.

### Step 3: (E)-3-(4-Bromophenyl)-1-(3-(tetrahydro-2H-pyran-2-yloxy)cyclobutyl)prop-2-en-1-one

The title compound was produced in analogy to example 8 step 2 from dimethyl 2-oxo-2-(3-(tetrahydro-2H-pyran-2-yloxy)cyclobutyl)ethylphosphonate and 4-bromobenzaldehyde. Yellow oil; MS: 365.0 [M+H]⁺.

### Step 4: 3-(4-Bromophenyl)-1-(3-(tetrahydro-2H-pyran-2-yloxy)cyclobutyl-3-o-tolylpropan-1-one

The title compound was produced in analogy to example 8, step 3 from (E)-3-(4-bromophenyl)-1-(3-(tetrahydro-2H-pyran-2-yloxy)cyclobutyl)prop-2-en-1-one and o-tolyl magnesium bromide. Colorless gum, MS: 474.2 [M+NH₄]⁺.

### Step 5: 3-(4-Bromophenyl)-1-(3-hydroxycyclobutyl-3-o-tolylpropan-1-one

The title compound was produced in analogy to example 13, step 5 from 3-(4-bromophenyl)-1-(3-(tetrahydro-2H-pyran-2-yloxy)cyclobutyl)-3-o-tolylpropan-1-one. Light yellow oil, MS: 373.2 [M+H]⁺.

### Step 6: (E-3-(4-Bromophenyl)-1-(3-hydroxycyclobutyl)-3-o-tolylpropan-1-one oxime

The title compound was produced in analogy to examples 1 and 2, step 11 from 3-(4-bromophenyl)-1-(3-hydroxycyclobutyl)-3-o-tolylpropan-1-one. White solid, MS: 388.2 [M+H]⁺.

1. A compound of formula I, 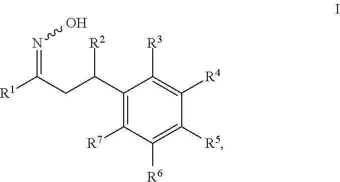
wherein
R¹ is C₄₋₇-cycloalkyl, said cycloalkyl being unsubstituted or substituted by one, two or three groups independently selected from the group consisting of C₁₋₇-alkyl, hydroxy, oxo, dioxo and C₁₋₇-alkylcarbonyl; or
heterocyclyl, said heterocyclyl having 4 to 7 ring atoms, comprising one, two or three heteroatoms selected from N, O and S and being unsubstituted or substituted by one, two or three groups independently selected from the group consisting of C₁₋₇-alkyl, hydroxy, oxo, dioxo, and C₁₋₇-alkylcarbonyl;
R² is selected from the group consisting of C₁₋₇-alkyl,
C₃₋₇-cycloalkyl, C₂₋₇-alkenyl, halogen-C₁₋₇-alkyl,
unsubstituted phenyl or phenyl substituted by one, two or three groups independently selected from the group consisting of C₁₋₇-alkyl, halogen, halogen-C₁₋₇-alkyl, halogen-C₁₋₇-alkoxy and C₁₋₇-alkylsulfonyl, and
heteroaryl, said heteroaryl being unsubstituted or substituted by C₁₋₇-alkyl or oxo,
R³ and R⁷ are independently from each other selected from the group consisting of hydrogen, halogen and C₁₋₇-alkyl; and
R⁴, R⁵ and R⁶ are independently selected from the group consisting of
hydrogen,
halogen, halogen-C₁₋₇-alkyl,
cyano, cyano-C₁₋₇-alkyl,
C₁₋₇-alkyl, C₁₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, caboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkylsulfonyl, C₁₋₇-alkylsulfonyloxy,
C₁₋₇-alkylsulfonylamino, C₃₋₇-cycloalkylsulfonylamino,
aminosulfonyl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₇-alkyl-amino,
hydroxy-C₁₋₇-alkyl-C₁₋₇-alkyl-amino, an amino acid attached through the amino group of the amino acid,
C₃₋₇-cycloalkyl-amino, wherein C₃₋₇-cycloalkyl is unsubstituted or substituted by hydroxy,
hydroxy-C₁₋₇-alkyl or carboxyl,
carboxyl-C₁₋₇-alkyl-aminocarbonyl, carboxyl-C₁₋₇-alkyl-(C₁₋₇-alkyl)aminocarbonyl, C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl,
hydroxy-C₁₋₇-alkyl-C₁₋₇-alkyl-aminocarbonyl, halogen-hydroxy-C₁₋₇-alkyl-aminocarbonyl, C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl-C₁₋₇-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₁₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahhydropyranyl, carboxyl, carboxyl-C₁₋₇-alkyl, C₁₋₇-alkoxy-C₁₋₇-alkyl or C₁₋₇-alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl,
C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl,
tetrazolyl-C₁₋₇-alkyl-aminocarbonyl and carboxyl-C₁₋₇-alkyl-aminocarbonyl;
or a pharmaceutically acceptable salt thereof.

2. The compound according to claim 1, wherein R¹ is heterocyclyl, said heterocyclyl having 4 to 7 ring atoms, comprising one, two or three heteroatoms selected from N, O and S and being unsubstituted or substituted by one, two or three groups independently selected from the group consisting of C₁₋₇-alkyl, hydroxy, oxo, dioxo, and C₁₋₇-alkylcarbonyl.

3. The compound according to claim 1, wherein R¹ is heterocyclyl, said heterocyclyl being selected from the group consisting of piperidinyl, tetrahydropyranyl and tetrahydrothiopyranyl and being unsubstituted or substituted by one, two or three groups independently selected from the group consisting of C₁₋₇-alkyl, hydroxy, oxo, dioxo, and C₁₋₇-alkylcarbonyl.

4. The compound according to claim 1, wherein R¹ is C₄₋₇-cycloalkyl, said cycloalkyl being substituted by one, two or three groups independently selected from the group consisting of C₁₋₇-alkyl, hydroxy, oxo, dioxo, and C₁₋₇-alkylcarbonyl.

5. The compound according to claim 1, wherein R¹ is selected from the group consisting of 1-methyl-2-oxo-piperidin-3-yl, 1-acetyl-piperidin-4-yl, tetrahydro-2H-pyran-4-yl, tetrahydro-2H-thiopyran-4-yl, 1,1-dioxo-hexahydro-thiopyran-4-yl, 3-hydroxycyclobutyl, 4-hydroxycyclohexyl, 4-oxocyclohexyl and 4-hydroxy-4-methylcyclohexyl.

6. The compound according to claim 1, wherein R² is unsubstituted phenyl or phenyl substituted by one, two or three groups independently selected from the group consisting of C₁₋₇-alkyl, halogen, halogen-C₁₋₇-alkyl, halogen-C₁₋₇-alkoxy and C₁₋₇-alkylsulfonyl.

7. The compound according to claim 1, wherein R² is 2-methylphenyl.

8. The compound according to claim 1, wherein R³ and R⁷ are hydrogen.

9. The compound according to claim 1, wherein R⁵ is selected from the group consisting of halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl,
carboxyl-C₁₋₇-alkoxy,
tetrazolyl,
C₁₋₇-alkoxycarbonyl,
C₁₋₇-alkylsulfonyl, C₁₋₇-alkylsulfonyloxy,
C₁₋₇-alkylsulfonylamino, C₃₋₇-cycloalkylsulfonylamino,
aminosulfonyl, (C₁₋₇-alkyl)-aminosulfonyl, di-(C₁₋₇-alkyl)-aminosulfonyl,
heterocyclylsulfonyl,
C₁₋₇-alkyl-amino, di-(C₁₋₇-alkyl)-amino, C₁₋₇-alkoxy-C₁₋₇-alkyl-amino,
C₁₋₇-alkoxy-C₁₋₇-alkyl-C₁₋₇-alkyl-amino, C₁₋₇-alkoxy-halogen-C₁₋₇-alkyl-amino,
hydroxy-C₁₋₇-alkyl-C₁₋₇-alkyl-amino, an amino acid attached through the amino group of the amino acid,
C₃₋₇-cycloalkyl-Amino, wherein C₃₋₇-cycloalkyl is unsubstituted or substituted by hydroxy,
hydroxy-C₁₋₇-alkyl or carboxyl,
carboxyl-C₁₋₇-alkyl-aminocarbonyl, carboxyl-C₁₋₇-alkyl-(C₁₋₇-alkyl)-aminocarbonyl,
C₁₋₇-alkoxycarbonyl-C₁₋₇-alkyl-aminocarbonyl,
C₁₋₇-alkyl-aminocarbonyl, di-(C₁₋₇-alkyl)-aminocarbonyl,
C₁₋₇-alkylsulfonyl-C₁₋₇-alkyl-aminocarbonyl,
halogen-C₁₋₇-alkyl-aminocarbonyl, hydroxy-C₁₋₇-alkyl-aminocarbonyl,
hydroxy-C₁₋₇-alkyl-C₁₋₇-alkyl-aminocarbonyl, halogen-hydroxy-C₁₋₇-alkyl-aminocarbonyl,
C₁₋₇-alkoxy-C₁₋₇-alkyl-aminocarbonyl,
C₃₋₇-cycloalkylaminocarbonyl, wherein C₃₋₇-cycloalkyl is unsubstituted or substituted by hydroxy, hydroxy-C₁₋₇-alkyl or carboxyl,
heterocyclyl-aminocarbonyl, wherein heterocyclyl is unsubstituted or substituted by C₁₋₇-alkyl or oxo,
heterocyclyl-C₁₋₇-alkyl-aminocarbonyl, wherein heterocyclyl is unsubstituted or substituted by C₁₋₇-alkyl or oxo,
hydroxy-C₁₋₇-alkyl-aminocarbonyl-C₁₋₇-alkyl,
C₁₋₇-alkoxycarbonyl)-C₁₋₇-alkyl,
di-(C₁₋₇-alkoxycarbonyl)-C₁₋₇-alkyl,
C₁₋₇-alkylcarbonylamino-C₁₋₇-alkylaminocarbonyl,
C₁₋₇-alkylcarbonylamino, carboxyl-C₁₋₇-alkylcarbonylamino,
C₁₋₇-alkoxycarbonyl-C₁₋₇-alkylcarbonylamino,
C₃₋₇-cycloalkyl, wherein C₃₋₇-cycloalkyl is unsubstituted or substituted by hydroxy, hydroxy-C₁₋₇-alkyl or carboxyl,
C₃₋₇-cycloalkyl-C₁₋₇-alkyl, wherein C₃₋₇-cycloalkyl is unsubstituted or substituted by hydroxy, hydroxy-C₁₋₇-alkyl or carboxyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, heterocyclylcarbonyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl or C₁₋₇-alkylsulfonyl,
heteroaryl, said heteroaryl being unsubstituted or substituted by C₁₋₇-alkyl, C₃₋₇-cycloalkyl, tetrahydropyranyl, carboxyl, carboxyl-C₁₋₇-alkyl, C₁₋₇-alkoxy-C₁₋₇-alkyl or C₁₋₇-alkoxycarbonyl,
phenyloxy, wherein phenyl is unsubstituted or substituted by one to three groups selected from halogen or carboxyl, and
phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl,
C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl,
tetrazolyl-C₁₋₇-alkyl-aminocarbonyl and carboxyl-C₁₋₇-alkyl-aminocarbonyl;
and R⁴ and R⁶ are hydrogen.

10. The compound according to claim 1 wherein R⁵ is selected from the group consisting of halogen, halogen-C₁₋₇-alkyl,
cyano, cyano-C₁₋₇-alkyl,
C₁₋₇-alkyl, C₃₋₇-alkenyl, C₁₋₇-alkynyl,
C₁₋₇-alkoxy, C₁₋₇-alkoxy-C₁₋₇-alkyl,
hydroxy, hydroxy-C₁₋₇-alkyl, hydroxy-C₃₋₇-alkenyl, hydroxy-C₃₋₇-alkynyl,
hydroxy-C₁₋₇-alkoxy,
carboxyl, carboxyl-C₁₋₇-alkyl, carboxyl-C₃₋₇-alkenyl, carboxyl-C₁₋₇-alkynyl, C₁₋₇-alkylsulfonyl,
heterocyclyl, said heterocyclyl being unsubstituted or substituted by C₁₋₇-alkyl, halogen, hydroxy, hydroxy-C₁₋₇-alkyl, C₁₋₇-alkoxy, oxo, carboxyl, carboxyl-C₁₋₇-alkyl, C₁₋₇-alkoxycarbonyl, aminocarbonyl, C₁₋₇-alkylsulfonyl, aminosulfonyl, C₁₋₇-alkylcarbonyl, carboxyl-C₁₋₇-alkyl-aminocarbonyl or hydroxysulfonyl-C₁₋₇-alkyl-aminocarbonyl, and phenyl, said phenyl being unsubstituted or substituted by one to three groups selected from the group consisting of halogen, C₁₋₇-alkyl, hydroxy, hydroxy-C₁₋₇-alkyl, cyano, cyano-C₁₋₇-alkyl, amino, C₁₋₇-alkoxy, carboxyl, carboxyl-C₁₋₇-alkyl,
C₁₋₇-alkoxy-carbonyl, tetrazolyl, carboxyl-C₁₋₇-alkyl-carbonylamino,
C₁₋₇-alkoxy-carbonyl-C₁₋₇-alkyl-carbonylamino, C₁₋₇-alkylsulfonyl,
C₁₋₇-alkyl-sulfonylamino, aminosulfonyl, C₁₋₇-alkyl-aminosulfonyl,
di-(C₁₋₇-alkyl)-aminosulfonyl, heterocyclylsulfonyl, C₁₋₇-alkoxycarbonyl-C₁₋₇-alkoxy, C₁₋₇-alkoxycarbonyl-C₁₋₇-alkyl-aminocarbonyl, carboxyl-C₁₋₇-alkyl-aminocarbonyl, C₁₋₇-alkoxycarbonyl-C₁₋₇-alkyl-carbonylamino-C₁₋₇-alkylsulfonyl,
phenyl-C₁₋₇-alkyl-aminocarbonyl, tetrazolyl-aminocarbonyl,
tetrazolyl-C₁₋₇-alkyl-aminocarbonyl and carboxyl-C₁₋₇-alkyl-aminocarbonyl;
and R⁴ and R⁶ are hydrogen.

11. The compound according to claim 1, wherein R⁵ is selected from the group consisting of halogen, halogen-C₁-alkyl,
carboxyl, carboxyl-C₁₋₇-alkyl, carboxyl-C₃₋₇-alkenyl, carboxyl-C₁₋₇-alkynyl,
C₁₋₇-alkylsulfonyl,
heterocyclyl, said heterocyclyl being unsubstituted or substituted by carboxyl or C₁₋₇-alkylsulfonyl, and
phenyl, said phenyl being unsubstituted or substituted by carboxyl;
and R⁴ and R⁶ are hydrogen.

12. The compound according to claim 1, selected from the group consisting of
(5-((R,E)-1-(hydroxyimino)-3-(4-(methylsulfonyl)phenyl)-3-o-tolylpropyl)-1-methylpiperidin-2-one,
5-((R,Z)-1-(hydroxyimino)-3-(4-(methylsulfonyl)phenyl)-3-o-tolylpropyl)-1-methylpiperidin-2-one,
5-[(R)-1-[(E)-hydroxyimino]-3-(4-methanesulfonyl-phenyl)-3-o-tolyl-propyl]-1-methyl-piperidin-2-one,
1-(4-((R,E)-3-(hydroxyimino)-3-(1-methyl-6-oxopiperidin-3-yl)-1-o-tolylpropyl)phenyl)piperidine-4-carboxylic acid,
sodium 1-(4-((R,E)-3-(hydroxyimino)-3-(1-methyl-6-oxopiperidin-3-yl)-1-o-tolylpropyl)phenyl)piperidine-4-carboxylate,
5-((R,E)-1-(hydroxyimino)-3-phenyl-3-o-tolylpropyl)-1-methylpiperidin-2-one,
4′-((1R,E)-3-(hydroxyimino)-3-(1-methyl-6-oxopiperidin-3-yl)-1-o-tolylpropyl)biphenyl-4-carboxylic acid,
(E)-1-(4-(3-(4-bromophenyl)-1-(hydroxyimino)-3-o-tolylpropyl)piperidin-1-yl)ethanone,
(E)-3-(4-bromophenyl)-1-(tetrahydro-2H-pyran-4-yl)-3-o-tolylpropan-1-one oxime,
(E)-3-(4-(methylsulfonyl)phenyl)-3-phenyl-1-(tetrahydro-2H-pyran-4-yl)propan-1-one oxime,
(E)-3-(4-bromophenyl)-1-(tetrahydro-2H-thiopyran-4-yl)-3-o-tolylpropan-1-one oxime,
3-(4-Bromo-phenyl)-1-(1,1-dioxo-hexahydro-thiopyran-4-yl)-3-o-tolyl-propan-1-one oxime,
(E)-3-(4-bromophenyl)-1-((1r,4r)-4-hydroxycyclohexyl)-3-o-tolylpropan-1-one oxime,
(E)-4-(3-(4-bromophenyl)-1-(hydroxyimino)-3-o-tolylpropyl)cyclohexanone,
(E)-1-(4-hydroxycyclohexyl)-3-(4-(methylsulfonyl)phenyl)-3-o-tolylpropan-1-one oxime,
(Z)-1-(4-hydroxycyclohexyl)-3-(4-(methylsulfonyl)phenyl)-3-o-tolylpropan-1-one oxime,
(E)-1-((r,4r)-4-hydroxy-4-methylcyclohexyl)-3-(4-(methylsulfonyl)phenyl)-3-o-tolylpropan-1-one oxime,
(E)-3-(4-bromophenyl)-1-(3-hydroxycyclobutyl)-3-o-tolylpropan-1-one oxime, and
pharmaceutically acceptable salts thereof.

13. A pharmaceutical composition comprising a compound according to claim 1 and a pharmaceutically acceptable carrier and/or adjuvant.

14. The compound of claim 1 wherein said compound is 5-[(R)-1-[(E)-Hydroxyimino]-3-(4-methanesulfonyl-phenyl)-3-o-tolyl-propyl]-1-methyl-piperidin-2-one or a pharmaceutically acceptable salt thereof.

15. The compound of claim 1 wherein said compound is 1-(4-((1R,E)-3-(hydroxyimino)-3-(1-methyl-6-oxopiperidin-3-yl)-1-o-tolylpropyl)phenyl)piperidine-4-carboxylic acid or a pharmaceutically acceptable salt thereof.

16. The compound of claim 15 wherein said compound is a sodium salt.
